(19) United States
(12) Patent Application Publication
(10) Pub. No.: US 2021/0189334 A1
(43) Pub. Date: Jun. 24, 2021

(54) Corneal Epithelial Cells and Their Products for Treating Corneal Diseases

(71) Applicant: Texas Tech University System, Lubbock, TX (US)

(72) Inventors: Hiranmoy Das, Upper Arlington, OH (US); Sloan W. Rush, Amarillo, TX (US)

(21) Appl. No.: 17/152,013

(22) Filed: Jan. 19, 2021

Related U.S. Application Data
(63) Continuation-in-part of application No. PCT/US2019/042525, filed on Jul. 19, 2019.
(60) Provisional application No. 62/700,639, filed on Jul. 19, 2018.

(51) Int. Cl. C12N 5/079; C12N 5/00; A61K 45/06; A61K 35/30

(52) U.S. Cl. CPC C12N 5/0621; A61K 35/30; A61K 45/06; C12N 5/0031

(57) ABSTRACT

The present invention includes compositions and method of generating human corneal epithelial stem cells, a human corneal epithelial stem cell supernatant, or both, the method comprising: wetting and mincing a corneal epithelial sample in a media, drying the minced corneal epithelial sample until sample edges are adhered to a substrate, adding a growth media comprising fetal bovine serum or human serum to the minced corneal epithelial sample without dislodging the minced corneal epithelial sample from the substrate with an amount of media that permits at least a portion of the minced corneal epithelial sample to be in contact with air, culturing the minced corneal epithelial sample for one or more days, changing the growth media to a media comprising a human corneal growth supplement (HCGS) with no fetal bovine serum, culturing the cells to grow human corneal epithelial stem cells, a human corneal epithelial stem cell supernatant, or both.

### CROSS-REFERENCE TO RELATED APPLICATIONS

This application claims priority to and is a continuation-in-part of International Application No. PCT/US2019/042525, filed Jul. 19, 2019, which claims priority to U.S. Provisional Application Ser. No. 62/700,639, filed Jul. 19, 2018, the entire contents of which are incorporated herein by reference.

### STATEMENT OF FEDERALLY FUNDED RESEARCH

None.

### TECHNICAL FIELD OF THE INVENTION

The present invention relates in general to the field of novel methods of making and using corneal epithelial cells and their products for treating corneal diseases.

### BACKGROUND OF THE INVENTION

Without limiting the scope of the invention, its background is described in connection with treatments of the eye.

Current therapies for dry eye and corneal ulcers include anti-inflammatory and/or lubricious eye drops and/or albumin patches as a delivery vehicle for pharmaceuticals. Current experimental therapies include transplanted limbic and/or corneal epithelial stem cells.

One such method is taught in U.S. Pat. No. 9,574,171, issued to Itskovitz-Eldor, et al., entitled “Methods of generating corneal cells and cell populations comprising same”. Briefly, these inventors are said to teach a method of generating a population of corneal epithelial by culturing human pluripotent stem cells in corneal fibroblast-conditioned medium on a solid surface comprising an extracellular matrix component and generating the population of corneal epithelial cells. Isolated cell populations and corneal tissues are also said to be disclosed.

Another such method is taught in U.S. Pat. No. 6,984,622, issued to Fleiszig and McNamara, entitled “Use of lipopolysaccharides to manage corneal infections and wounds”. Briefly, these inventors teach that the antibiotic polypeptide β-defensin-2 (hBD-2) is expressed in the eye, and is useful for treating ocular wounds. hBD-2 is increased in the eye upon exposure to lipopolysaccharides (LPS). Administration of LPS to the eye is said to provide a useful method for increasing the amount of this antibiotic peptide in the eye. The LPS is obtained from Pseudomonas aeruginosa strain PAO1.

U.S. Pat. Nos. 5,585,265, 5,672,498, and 5,786,201 are said disclose inventions directed to the production of human corneal epithelial cell strains with extended lifespans. Although the cell strains are derived from human corneal epithelial cells, they are immortalized cell lines established by viral infection or plasmid transfection. These cell strains may be useful for in vitro experiments for studying the effects of chemicals and drugs on the human eye, however, these continuous cell strains are inappropriate for human transplantation because of the obvious risk of infection and rejection problems.

Another such method is taught in U.S. Patent Publication No. US20020039788A1 filed by Isseroff and Schwab, entitled “Corneal epithelial graft composites”. Accordingly to the applicants, their invention is directed to a bioengineered composite graft for the treatment of a damaged or diseased corneal epithelial surface wherein the corneal epithelial composite graft comprises ex vivo corneal epithelial stem cells cultured on an extracellular carrier matrix, the methods of making and using the corneal epithelial composite graft.

U.S. Patent Application No. US20050186672A1 filed by Mahadeorao and Devi, entitled “Tissue system with undifferentiated stem cells derived from corneal limbus”. These applicants teach a method comprised of (a) isolating corneal limbal tissue from a donor; (b) culturing the corneal limbal tissue to expand corneal limbal cells in culture; (c) isolating a population of limbal stem cells from the cultured corneal limbal cells by sorting the corneal limbal cells to select for one or more stem cell-specific surface markers, wherein the stem cell-specific surface marker is expressed by undifferentiated stem cells (USCs); (d) culturing the isolated population of USCs to generate the tissue system.

However, despite advances in these areas many patients continue to suffer despite these treatments.

### SUMMARY OF THE INVENTION

In one embodiment, the present invention includes a method of generating a population of human corneal epithelial stem cells or a human corneal epithelial stem cell supernatant comprising: wetting and mincing a corneal epithelial sample in a media; drying the minced corneal epithelial sample until sample edges are adhered to a substrate; adding a growth media comprising fetal bovine serum or human serum; culturing the minced corneal epithelial sample for one or more days; changing the growth media to a serum-free media comprising a human corneal growth supplement (HCGS) with no fetal bovine serum or human serum; culturing the cells for 1 to 3 weeks, and optionally changing the serum-free media every three days; and harvesting the human corneal epithelial stem cells, the human corneal epithelial stem cell supernatant, or both. In one aspect, method further comprises: dissociating a population of human corneal epithelial cells isolated to generate a population of dissociated human corneal epithelial cells; and culturing the dissociated human corneal epithelial cells in a media comprising fetal bovine serum or human serum for 1 to 3 weeks or until the human corneal epithelial stem cells, limbal stem cells, or both grow. In another aspect, the dissociated human corneal epithelial cell culture media comprises αMEM with 20% fetal bovine serum (FBS) or human serum until the cells adhere and start propagating changing the media every 2-3 days; and culturing the adhered, propagating human corneal epithelial cells in a media comprising a human corneal growth supplement (HCGS) with no FBS or human serum until cells reach confluence or near confluence to grow human corneal epithelial stem cells, produce a human corneal epithelial stem cell supernatant, or both. In another aspect, the e drying step induces adhesion of the tissue edges only, or in the step of adding a growth media comprising fetal bovine serum or human serum to the minced corneal epithelial sample the minced corneal epithelial sample is not dislodging from the substrate with an amount of media that permits at least a portion of the minced corneal epithelial sample to be in contact with air. In another aspect, the method further comprises repeating the step of harvesting the human corneal epithelial stem cells and reseeding the cells in serum-free media comprising HCGS one or more times to 70, 75, 80, 85, 90, or 95% percent confluency, and optionally reseeding in double the volume the serum-free media comprising HCGS. In another aspect, the method further comprises splitting and re-plating the human corneal epithelial cells when they reach confluence or near confluence in the HCGS media. In another aspect, the method further comprises repeating the step of harvesting the human corneal epithelial stem cells one or more times, by splitting the cells and re-plating prior to repeating, to obtain additional cells. In another aspect, the supernatant comprises glycocalyx, microvesicles, exosomes, microRNA, growth factors, cytokines, and inflammatory inhibitors. In another aspect, the corneal epithelial sample is autologous or cadaveric. In another aspect, the method further comprises treating a subject with a disease or disorder of the eye selected from: severe dry eye disease, a corneal epithelial disease or disorder selected from at least one of: including but not limited to: mechanical trauma (e.g. fingernail scratch, contact lens overuse, foreign body in the lid/fornices, trichiasis/distichiasis, chemical exposure); chronic exposure to air (e.g. neurotrophic diseases causing incomplete lid closure such as cranial nerve VII palsy, restrictive eyelid diseases, proptosis, decreased consciousness in drug abuse or comatose state, blepharoplasty, lagophthalmos); ultraviolet burns (e.g. welding, prolonged sun exposure off reflective surfaces); local corneal dryness and systemic disorders leading to corneal dryness, dry eye syndrome, thyroid eye disease, Sjogren's syndrome, vitamin A deficiency; limbal stem cell deficiency, failure to regenerate epithelial cells, occurs from a variety of causes chemical burns, post ocular surgery, ocular autoimmune degenerations); topical anesthetic abuse; neurotrophic keratopathy, corneal hypoesthesia or anesthesia caused, most frequently, by damage to the trigeminal nerve, also human simplex virus (HSV), varicella-zoster virus (VZV), and topical drop toxicity, blepharitis, meibomian gland dysfunction, chronic ocular surface disease, neurotrophic keratoconjunctivitis, corneal ulcer, marginal keratitis, peripheral ulcerative keratitis, acute keratitis, chronic keratitis, acute conjunctivitis, chronic conjunctivitis, anterior scleritis, corneal abrasion, corneal edema, recurrent corneal erosion, delayed corneal epithelial wound healing, corneal postoperative healing, or corneal neovascularization.

In another embodiment, the present invention includes a method for making a corneal epithelial stem cells, a corneal epithelial stem cell culture supernatant, or both, comprising: wetting and mincing a corneal epithelial sample in a media; drying the minced corneal epithelial sample until sample edges are adhered to a substrate; adding a growth media comprising fetal bovine serum or human serum; culturing the minced corneal epithelial sample for one or more days; changing the growth media to a serum-free media comprising a human corneal growth supplement (HCGS) with no fetal bovine serum or human serum; culturing the cells for 1 to 3 weeks, and optionally changing the serum-free media every three days; harvesting the human corneal epithelial stem cells; washing the cells with PBS or HBSS; culturing overnight in PBS or HBSS only; collecting the corneal epithelial stem cell supernatant; centrifuging the supernatant to remove any non-adherent cells; and harvesting more human corneal epithelial stem cells, the human corneal epithelial stem cell supernatant, or both, one or more times by re-culturing the surviving adherent or non-adherent cells or both.

In another embodiment, the present invention includes a method of treating a disease or disorder of the eye in a patient, comprising: administering to the patient a composition comprising a population of human corneal epithelial cells or a corneal epithelial stem cell supernatant, or both, made by a method comprising: wetting and mincing a corneal epithelial sample in a media; drying the minced corneal epithelial sample until sample edges are adhered to a substrate; adding a growth media comprising fetal bovine serum or human serum; culturing the minced corneal epithelial sample for one or more days; changing the growth media to a serum-free media comprising a human corneal growth supplement (HCGS) with no fetal bovine serum or human serum; culturing the cells for 1 to 3 weeks, and optionally changing the serum-free media every three days; harvesting the human corneal epithelial stem cells, the corneal epithelial stem cell supernatant, or both; and providing the patient with the human corneal epithelial stem cells, the corneal epithelial stem cell supernatant, or both to treat the disease or disorder of the eye. In one aspect, the method further comprises: dissociating a population of human corneal epithelial cells isolated to generate a population of dissociated human corneal epithelial cells; and culturing the dissociated human corneal epithelial cells in a media comprising fetal bovine serum or human serum for 1 to 3 weeks or until the human corneal epithelial stem cells, limbal stem cells, or both grow. In another aspect, the disease or disorder of the eye is a corneal epithelial disease or disorder selected from at least one of: including but not limited to: severe dry eye disease, mechanical trauma (e.g. fingernail scratch, contact lens overuse, foreign body in the lid/fornices, trichiasis/distichiasis, chemical exposure); chronic exposure to air (e.g. neurotrophic diseases causing incomplete lid closure such as cranial nerve VII palsy, restrictive eyelid diseases, proptosis, decreased consciousness in drug abuse or comatose state, blepharoplasty, lagophthalmos); ultraviolet burns (e.g. welding, prolonged sun exposure off reflective surfaces); local corneal dryness and systemic disorders leading to corneal dryness, dry eye syndrome, thyroid eye disease, Sjogren's syndrome, vitamin A deficiency; limbal stem cell deficiency, failure to regenerate epithelial cells, occurs from a variety of causes chemical burns, post ocular surgery, ocular autoimmune degenerations); topical anesthetic abuse; neurotrophic keratopathy, corneal hypoesthesia or anesthesia caused, most frequently, by damage to the trigeminal nerve, also human simplex virus (HSV), varicella-zoster virus (VZV), and topical drop toxicity, blepharitis, meibomian gland dysfunction, chronic ocular surface disease, neurotrophic keratoconjunctivitis, corneal ulcer, marginal keratitis, peripheral ulcerative keratitis, acute keratitis, chronic keratitis, acute conjunctivitis, chronic conjunctivitis, anterior scleritis, corneal abrasion, corneal edema, recurrent corneal erosion, delayed corneal epithelial wound healing, corneal postoperative healing, or corneal neovascularization. In another aspect, the disease or disorder of the cornea leads to an injury such as ulceration of the corneal epithelium with possible erosion into the stromal areas. In another aspect, the supernatant comprises glycocalyx, microvesicles, exosomes, microRNA, growth factors, cytokines, and inflammatory inhibitors. In another aspect, the corneal epithelial sample is autologous or cadaveric. In another aspect, the human corneal epithelial cells, the corneal epithelial stem cell supernatant, or both are administered 1, 2, 3, 4, 5, or 6 times daily in each affected eye.

In another embodiment, the present invention includes a formulation comprising a human corneal epithelial stem cell supernatant, corneal epithelial stem cells, or both, made by a method comprising: wetting and mincing a corneal epithelial sample in a media; drying the minced corneal epithelial sample until sample edges are adhered to a substrate; adding a growth media comprising fetal bovine serum or human serum; culturing the minced corneal epithelial sample for one or more days; changing the growth media to a serum-free media comprising a human corneal growth supplement (HCGS) with no fetal bovine serum or human serum; culturing the cells for 1 to 3 weeks, and optionally changing the serum-free media every three days; and harvesting the human corneal epithelial stem cells, the human corneal epithelial stem cell supernatant, or both. In one aspect, the human corneal epithelial stem cell supernatant, corneal epithelial stem cells, or both are formulated into eye drops, serum, gel, or spray. In another aspect, the human corneal epithelial stem cell supernatant, corneal epithelial stem cells, or both are combined with a biocompatible or biodegradable substrate, hydrogel, collagen, polymer, sheet or a membrane. In another aspect, the formulation further comprises one or more active agents including an amniotic fluid, an antibiotic, an anti-viral agent, a hormone, a growth factor, a cytokine, a chemokine, a lymphokine, an antibody or fragment thereof, a peptide, a protein, a carbohydrate, or a nucleic acid.

In another embodiment, the present invention includes a human corneal epithelial stem cell or supernatant thereof made by a method comprising: wetting and mincing a corneal epithelial sample in a media; drying the minced corneal epithelial sample until sample edges are adhered to a substrate; adding a growth media comprising fetal bovine serum or human serum; culturing the minced corneal epithelial sample for one or more days; changing the growth media to a serum-free media comprising a human corneal growth supplement (HCGS) with no fetal bovine serum or human serum; culturing the cells for 1 to 3 weeks, and optionally changing the serum-free media every three days; and harvesting the human corneal epithelial stem cells, the human corneal epithelial stem cell supernatant, or both. In one aspect, the human corneal epithelial stem cells or supernatant, further comprises the step of differentiating the human corneal epithelial stem cell into human mature corneal epithelial cells. In another aspect, the human corneal epithelial stem cells or supernatant, further comprises the step of adding the stem cells, the supernatant, or both into or a biocompatible or biodegradable drop, substrate, hydrogel, collagen, polymer, sheet or membrane.

In one embodiment, the present invention includes a method of generating a population of human corneal epithelial stem cells comprising: wetting and mincing a corneal epithelial sample in a media; drying the minced corneal epithelial sample until sample edges are adhered to a substrate; adding a growth media comprising fetal bovine serum or human serum to the minced corneal epithelial sample without dislodging the minced corneal epithelial sample from the substrate with an amount of media that permits at least a portion of the minced corneal epithelial sample to be in contact with air; culturing the minced corneal epithelial sample for one or more days; changing the growth media to a media comprising a human corneal growth supplement (HCGS) with no fetal bovine serum; culturing the cells for 1 to 3 weeks; and harvesting the human corneal epithelial stem cells. In one aspect, the method further comprises: dissociating a population of human corneal epithelial cells isolated to generate a population of dissociated human corneal epithelial cells; culturing the dissociated human corneal epithelial cells in a media comprising fetal bovine serum or human serum until the cells grow (typically 1 to 3 weeks). In another aspect, the dissociated human corneal epithelial cell culture media comprises αMEM with 20% fetal bovine serum (FBS) or human serum until the cells adhere and start propagating changing the media every 2-3 days; and culturing the adhered, propagating human corneal epithelial cells in a media comprising a human corneal growth supplement (HCGS) with no FBS until cells reach confluence or near confluence to grow human corneal epithelial stem cells, produce a human corneal epithelial stem cell supernatant, or both. In another aspect, the drying step induces adhesion of the tissue edges only. In another aspect, the method further comprises the step of replacing the growth media comprising HCGS every 2 to 3 days. In another aspect, the method further comprises the step of re-plating the human corneal epithelial cells every 2 or more days in the HCGS media. In another aspect, the method further comprises the step of repeating the step of harvesting the human corneal epithelial stem cells one or more times, by splitting the cells and re-plating prior to repeating, to obtain additional cells. In another aspect, the method further comprises the step of harvesting a corneal epithelial stem cell supernatant. In another aspect, the supernatant comprises glycocalyx, microvesicles, exosomes, microRNA, growth factors, cytokines, and inflammatory inhibitors. In another aspect, the corneal epithelial sample is autologous.

In another embodiment, the present invention includes a method of generating a population of human corneal epithelial stem cells comprising: wetting and mincing a corneal epithelial sample in a media; drying the minced corneal epithelial sample until sample edges are adhered to a substrate; adding a growth media comprising fetal bovine serum or human serum to the minced corneal epithelial sample without dislodging the minced corneal epithelial sample from the substrate with an amount of media that permits at least a portion of the minced corneal epithelial sample to be in contact with air; culturing the minced corneal epithelial sample for one or more days; changing the growth media to a media comprising a human corneal growth supplement (HCGS) with no fetal bovine serum; culturing the cells for 1 to 3 weeks; harvesting the human corneal epithelial stem cells; dissociating a population of human corneal epithelial cells isolated to generate a population of dissociated human corneal epithelial cells; and culturing the dissociated human corneal epithelial cells in a media comprising fetal bovine serum or human serum until the cells grow. In one aspect, the method further comprises the step of replacing the growth media comprising HCGS every 2 to 3 days. In another aspect, the method further comprises the step of splitting and re-plating the human corneal epithelial cells every 2 or more days in the HCGS media. In another aspect, the method further comprises the step of harvesting a corneal epithelial stem cell supernatant. In another aspect, the corneal epithelial sample is autologous. In another aspect, the drying step induces adhesion of the tissue edges only.

In another embodiment, the present invention includes a method for making a corneal epithelial stem cell culture supernatant comprising: obtaining a source of corneal epithelial stem cells according to claim 1; washing the cells with PBS or HBSS; culturing overnight in PBS or HBSS only; collecting the corneal epithelial stem cell supernatant; centrifuging the supernatant to remove any non-adherent cells; optionally harvesting more human corneal epithelial stem cell supernatant one or more times by re-culturing the adherent cells but without dissociating the cells; dissociating a population of human corneal epithelial cells isolated to generate a population of dissociated human corneal epithelial cells; and culturing the dissociated human corneal epithelial cells in a media comprising fetal bovine serum or human serum until the cells grow. In one aspect, the supernatant comprises glycocalyx, microvesicles, exosomes, microRNA, growth factors, cytokines, and inflammatory inhibitors. In another aspect, the drying step induces adhesion of the tissue edges only.

In another embodiment, the present invention includes a method of treating a disease or disorder of the eye in a patient, comprising: administering to the patient a composition comprising a population of human corneal epithelial cells or a corneal epithelial stem cell supernatant, or both made by a method comprising: wetting and mincing a corneal epithelial sample in a media; drying the minced corneal epithelial sample until sample edges are adhered to a substrate; adding a growth media comprising fetal bovine serum or human serum to the minced corneal epithelial sample without dislodging the minced corneal epithelial sample from the substrate with an amount of media that permits at least a portion of the minced corneal epithelial sample to be in contact with air; culturing the minced corneal epithelial sample for one or more days; changing the growth media to a media comprising a human corneal growth supplement (HCGS) with no fetal bovine serum; culturing the cells for 1 to 3 weeks; and harvesting the human corneal epithelial stem cells, the corneal epithelial stem cell supernatant, or both; and providing the patient with the human corneal epithelial stem cells, the corneal epithelial stem cell supernatant, or both to treat the disease or disorder of the eye. In one aspect, method further comprises dissociating a population of human corneal epithelial cells isolated to generate a population of dissociated human corneal epithelial cells; and culturing the dissociated human corneal epithelial cells in a media comprising fetal bovine serum or human serum until the cells for 1 to 3 weeks or until the cells grow. In another aspect, the disease or disorder of the eye is a disease or disorder of the cornea. In another aspect, the disease or disorder of the eye is a corneal epithelial disease or disorder selected from at least one of: including but not limited to: mechanical trauma (e.g. fingernail scratch, contact lens overuse, foreign body in the lid/fornices, trichiasis/distichiasis, chemical exposure); chronic exposure to air (e.g. neurotrophic diseases causing incomplete lid closure such as cranial nerve VII palsy, restrictive eyelid diseases, proptosis, decreased consciousness in drug abuse or comatose state, blepharoplasty, lagophthalmos); ultraviolet burns (e.g. welding, prolonged sun exposure off reflective surfaces); local corneal dryness and systemic disorders leading to corneal dryness, dry eye syndrome, thyroid eye disease, Sjogren's syndrome, vitamin A deficiency; limbal stem cell deficiency, failure to regenerate epithelial cells, occurs from a variety of causes chemical burns, post ocular surgery, ocular autoimmune degenerations); topical anesthetic abuse; neurotrophic keratopathy, corneal hypoesthesia or anesthesia caused, most frequently, by damage to the trigeminal nerve, also human simplex virus (HSV), varicella-zoster virus (VZV), and topical drop toxicity, blepharitis, meibomian gland dysfunction, chronic ocular surface disease, neurotrophic keratoconjunctivitis, corneal ulcer, marginal keratitis, peripheral ulcerative keratitis, acute keratitis, chronic keratitis, acute conjunctivitis, chronic conjunctivitis, anterior scleritis, corneal abrasion, corneal edema, recurrent corneal erosion, delayed corneal epithelial wound healing, corneal postoperative healing, or corneal neovascularization. In another aspect, the disease or disorder of the cornea leads to an injury such as ulceration of the corneal epithelium with possible erosion into the stromal areas. In another aspect, the supernatant comprises glycocalyx, microvesicles, exosomes, microRNA, growth factors, cytokines, and inflammatory inhibitors. In another aspect, the corneal epithelial sample is autologous.

In another embodiment, the present invention includes a formulation comprising a human corneal epithelial stem cell supernatant, the supernatant made by a method comprising: wetting and mincing a corneal epithelial sample in a media; drying the minced corneal epithelial sample until sample edges are adhered to a substrate; adding a growth media comprising fetal bovine serum or human serum to the minced corneal epithelial sample without dislodging the minced corneal epithelial sample from the substrate with an amount of media that permits at least a portion of the minced corneal epithelial sample to be in contact with air; culturing the minced corneal epithelial sample for one or more days; changing the growth media to a media comprising a human corneal growth supplement (HCGS) with no fetal bovine serum; culturing the cells for 1 to 3 weeks; and harvesting the human corneal epithelial stem cell supernatant. In one aspect, the formulation is adapted into eye drops, serum, gel, or spray. In another aspect, the formulation is combined with a biocompatible or biodegradable: substrate, hydrogel, collagen, polymer, sheet or a membrane. In another aspect, the formulation further comprises one or more active agents including an amniotic fluid, an antibiotic, an anti-viral agent, a hormone, a growth factor, a cytokine, a chemokine, a lymphokine, an antibody or fragment thereof, a peptide, a protein, a carbohydrate, or a nucleic acid. In another aspect, the drying step induces adhesion of the tissue edges only.

A human corneal epithelial stem cell made by a method comprising: wetting and mincing a corneal epithelial sample in a media; drying the minced corneal epithelial sample until sample edges are adhered to a substrate; adding a growth media comprising fetal bovine serum or human serum to the minced corneal epithelial sample without dislodging the minced corneal epithelial sample from the substrate with an amount of media that permits at least a portion of the minced corneal epithelial sample to be in contact with air; culturing the minced corneal epithelial sample for one or more days; changing the growth media to a media comprising a human corneal growth supplement (HCGS) with no fetal bovine serum; culturing the cells for 1 to 3 weeks; and harvesting the human corneal epithelial stem cells. In another aspect, human corneal epithelial stem cells are differentiated into at least one of: human corneal epithelial stem cells (hCEpiSC), human corneal epithelial precursor cells (hCEpiPC), dry eye, corneal ulcer, or limbal stem cells. In another aspect, human corneal epithelial stem cells are added into or a biocompatible or biodegradable drop, substrate, hydrogel, collagen, polymer, sheet or membrane. In another aspect, the drying step induces adhesion of the tissue edges only.

### BRIEF DESCRIPTION OF THE DRAWINGS

For a more complete understanding of the features and advantages of the present invention, reference is now made to the detailed description of the invention along with the accompanying figures and in which:

FIG. 1 is a micrograph of an image of short-term survival (variant of) corneal epithelial stem cells, showing their expansion over 10 to 20 days.

FIG. 2 is a micrograph of an image of long-term survival (variant of) corneal epithelial stem cells, showing their expansion over 3 to 6 months.

FIG. 3 is a flow chart of one embodiment of one method of the present invention.

FIGS. 4A to 4C show Corneal Epithelial Stem Cell-Derived Therapy for Dry Eye Disease. Mean Patient Reported Outcome Measurements of over Various Time Intervals during the 12 Week Treatment Period for (FIG. 4A) Standardized Patient Evaluation of Eye Dryness (SPEED™) Questionnaire, (FIG. 4B) Ocular Surface Disease Index (OSDI©) Score and (FIG. 4C) Visual Analog Scale (VAS).

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not limit the invention, except as outlined in the claims.

The present inventors have isolated, grown, and increased corneal epithelial stem cells (hCEpiSCs) from a severely limited supply to a large available supply, for use in research and in patient care. In particular, the present inventors were able to grow and identify two types of hCEpiSC's: short-term surviving cells and long-term surviving cells. For the long-term surviving cells, the present inventors have confirmed their morphology in vitro and isolated cell products. These cells and their products may provide unique opportunities for improved dry eye treatment and to treat other corneal diseases.

Currently, most dry eye patients continue to suffer and simply settle for anything that only slightly reduces discomfort. The novel cells and methods of the present invention are precursors of the corneal epithelial cells and they secrete a key structure called the glycocalyx, which is implicated for maintaining healthy corneal epithelium. The present inventors can grow long-term surviving cells into large numbers, and obtain their supernatant, which include the key structural proteins, in particular, the glycocalyx and related factors. The present invention provides, for the first time, sufficient cells that secrete key cell secretory products (such as the glycocalyx) for use as a therapeutic. There are other uses for these stem cells and cell secretory products in patient treatment and for the development of novel agents to treat the eye.

In the prior art, e.g., U.S. Patent Publication No. 20020039788, these applicants are said to teach enriching the plurality of stem cells with an extracellular matrix protein composition, wherein the extracellular matrix protein composition comprises laminin, collagen, tenascin or a combination thereof. While the epithelial stem cells are said to be corneal epithelial stem cells, the step of isolating the plurality of stem cells further comprises: (i) obtaining a sample of tissue comprising the plurality of stem cells from the superior temporal limbus of the eye of the donor; (ii) washing the sample in a suitable solution or medium; and (iii) dissociating the plurality of stem cells to form a single cell suspension. The method is said to further comprise the step of: (i) adhering the plurality of stem cells in the single cell suspension to a surface coated with an extracellular matrix protein composition, wherein the extracellular matrix protein composition comprises laminin, collagen, tenascin, or a combination thereof. However, these applicants are using an extracellular matrix supplemented with proteins to grow the corneal limbal cells and as a carrier for the cells for placement on the corneal wound. The supplemental proteins are not derived from proteins secreted by the limbal cells. Further, the applicants isolate the cells by immediately dissociating the limbal tissue samples, culturing on a surface coated with one of their extracellular matrix components, then washing away all other cells, then dissociating the adherent cells and re-plating to expand the population. In sharp contrast, the method of the present invention begins with culturing the tissues and letting the stem cells migrate out of the tissue sample prior to dissociating and re-plating at a much later step.

U.S. Patent Application No. US20050186672A1 these applicants are said to teach a method comprised of (a) isolating corneal limbal tissue from a donor; (b) culturing the corneal limbal tissue to expand corneal limbal cells in culture; (c) isolating a population of limbal stem cells from the cultured corneal limbal cells by sorting the corneal limbal cells to select for one or more stem cell-specific surface markers, wherein the stem cell-specific surface marker is expressed by undifferentiated stem cells (USCs); (d) culturing the isolated population of USCs to generate the tissue system. Specifically, the prior art teaches that a preferred method of culturing the limbal tissue biopsies is to subject the explant to dry incubation for several minutes, either before or after placing the explant on an extracellular matrix or biocoated tissue culture plate. A small amount of culture medium is then added to the explant so that it sticks to the extracellular matrix or biocoated tissue culture surface. After several hours to a day, additional media is gently added and the explant is incubated for several days. This prior art differs significantly from the present invention for the following reasons. In the preset invention, the corneal tissue is harvested from dissected donor cornea, specifically from the epithelial layer of the cornea, thereby avoiding contamination with mesenchymal stem cells (MSCs) and/or stromal cells. In the prior art, they obtain their tissue from biopsies; hence, they must manage potential contamination with other cell types such as MSCs or stromal cells. In one embodiment (direct processing of tissue biopsy into a single cell suspension), the prior art teaches that it must carefully process their tissue samples with enzymatic dissociation to enable mechanical separation of the epithelial layer from the rest of the cornea biopsy with few MSCs or stromal cells contaminating their cultures (even with extensive processing, MSCs or stromal cells may still be present). By contrast, the tissue samples of the present invention are taken only from within the limbal zone, which avoids contamination with mesenchymal stem cells (MSCs) or stromal cells. In another difference, the prior art uses a drying method that requires first drying the tissue then inducing adhesion to a coated culture plate surface by wetting the tissue (i.e., wetting is a second step to induce adhesion). By contrast, the present invention begins with wetting the harvested tissue followed by a drying step that induces adhesion of the tissue edges only.

The epithelial stem cells can be transplanted or their products can be isolated and formulated into compositions and used in methods to treat corneal disorders and inflammation. These human corneal epithelial stem cells (hCEpiSCs) have been isolated and grown from a limited supply to a large enough volume for research and patient care. Differentiation of two hCEpiSCs are described herein, including both short term surviving cells and long term surviving cells. In vivo morphology of long term surviving cells have been completed and isolation of their product, glycocalyx, show potential in treatment of dry eye and other corneal diseases. Glycocalyx and its co-factors of the present invention can be used in the maintenance of healthy epithelial cells. Further research will be done to identify and isolate other potential therapeutic cells and secretory factors for treatment.

In one example, the present invention includes a method of making a Corneal Epithelial Stem Cell Culture and Corneal Epithelial Stem Cell Culture Supernatant. Briefly, the method includes a method of generating human corneal epithelial stem cells, a human corneal epithelial stem cell supernatant, or both, the method comprising: (1) obtaining a tissue sample from a human corneal limbal area, wetting and mincing the tissue, and plating on a tissue culture dish, allowing the edges to dry; (2) culturing the minced human corneal limbal tissues in a cell culture media (such as DMEM or αMEM with antibiotic(s) and antimycotic(s)) supplemented with fetal bovine serum (or its equivalent) for one or more days with an amount of media that permits at least a portion of the minced tissue to be in contact with air; and (3) culturing the tissue in a corneal epithelial cell culture media (such as human corneal epithelial cell culture media from Fisher Scientific) supplemented with human corneal epithelial cell growth supplement (HCGS) without fetal bovine serum or human serum for 1 to 3 weeks, changing the media every third day; and (4) dissociating cells and re-plating in a new culture dish with fresh media from step (3) above, and growing to confluence or near-confluence. Harvesting the cells directly generating and collecting supernatant, or re-culturing the cells to obtain additional cells, supernatant or both. The corneal epithelial stem cell culture supernatant can be made by: (1) washing the cells with PBS or HBSS and culturing overnight in PBS or HBSS only; and (2) collecting the corneal epithelial stem cell supernatant and centrifuging the supernatant to remove any non-adherent (floating) cells. The supernatant can then be used directly or frozen for later use. Either the human corneal epithelial stem cells, the human corneal epithelial stem cell supernatant, or both can be formulated into a therapeutic agent for the treatment of a variety of diseases, conditions, and syndromes of the eye. These diseases, conditions, and syndromes include corneal epithelial diseases, including but not limited to: mechanical trauma (e.g. fingernail scratch, contact lens overuse, foreign body in the lid/fornices, trichiasis/distichiasis, chemical exposure); chronic exposure to air (e.g. neurotrophic diseases causing incomplete lid closure such as cranial nerve VII palsy, restrictive eyelid diseases, proptosis, decreased consciousness in drug abuse or comatose state, blepharoplasty, lagophthalmos); ultraviolet burns (e.g. welding, prolonged sun exposure off reflective surfaces); local corneal dryness and systemic disorders leading to corneal dryness (e.g. dry eye syndrome, thyroid eye disease, Sjogren's syndrome, vitamin A deficiency); limbal stem cell deficiency (failure to regenerate epithelial cells, occurs from a variety of causes e.g. chemical burns, post ocular surgery, ocular autoimmune degenerations); topical anesthetic abuse; neurotrophic keratopathy (corneal hypoesthesia or anesthesia caused, most frequently, by damage to the trigeminal nerve, also human simplex virus (HSV), varicella-zoster virus (VZV), and topical drop toxicity, among others), dry eye disease, blepharitis, meibomian gland dysfunction, chronic ocular surface disease, neurotrophic keratoconjunctivitis, corneal ulcer, marginal keratitis, peripheral ulcerative keratitis, acute and chronic keratitis, acute and chronic conjunctivitis, anterior scleritis, corneal abrasion, corneal edema, recurrent corneal erosion, delayed corneal epithelial wound healing, corneal postoperative healing, corneal neovascularization. Those skilled in the art will realize that other eye diseases, conditions, and syndromes may also benefit from treatment with these corneal epithelial stem cells or their supernatant or both, since all or nearly all eye tissues have a similar origin (i.e., neural crest).

Techniques and compositions for making useful dosage forms using the present invention are described in one or more of the following references: Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 2007; Goodman and Gilman, eds., The Pharmacological Basis of Therapeutics, Tenth Edition, McGraw Hill, 2001; Remington's Pharmaceutical Sciences, 20th Ed., Lippincott Williams & Wilkins., 2000; Martindale, The Extra Pharmacopoeia, Thirty-Second Edition (The Pharmaceutical Press, London, 1999), and updates thereto; all of which are incorporated by reference, and the like, relevant portions incorporated herein by reference.

As used herein, the phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. For example, pharmaceutically acceptable carriers for administration of cells typically is a carrier acceptable for delivery by injection, and do not include agents such as detergents or other compounds that could damage the cells to be delivered. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations, particularly phosphate buffered saline solutions which are preferred for intraocular delivery.

Non-limiting examples of pharmaceutically acceptable carriers for delivery to the eye include, but are not limited to, suspension-type eye drops, eye wash, an eye gel, an eye cream, ointment, gel, liposomal dispersion, colloidal microparticle suspension, and the like, and other preparations known to those of skill in the art to be suitable for ocular administration. As such, the pharmaceutical compositions of the present invention containing human corneal epithelial stem cells, the human corneal epithelial stem cell supernatant, or both may be administered using commonly known devices configured for the delivery of the pharmaceutical compositions in the form of to the region surrounding the eye. An ocular insert may also include a biodegradable controlled release polymeric matrix, that can be implanted in the conjunctiva, sclera, pars plana, anterior segment, or posterior segment of the eye. The pharmaceutically acceptable carrier of the pharmaceutical composition of the invention may comprise a wide variety of non-active ingredients which are useful for formulation purposes and which do not materially affect the novel and useful properties of human corneal epithelial stem cells, the human corneal epithelial stem cell supernatant, or both.

The present invention may also include suitable thickeners known to those of ordinary skill in the art of ophthalmic formulation, e.g., cellulosic polymers such as methylcellulose (MC), hydroxyethylcellulose (HEC), hydroxypropylcellulose (HPC), hydroxypropylmethylcellulose (HPMC), and sodium carboxymethylcellulose (NaCMC), and other swellable hydrophilic polymers such as polyvinyl alcohol (PVA), hyaluronic acid or a salt thereof (e.g., sodium hyaluronate), and crosslinked acrylic acid polymers commonly referred to as “carbomers” that may or may not be biodegradable. The preferred amount of any thickener is such that a viscosity in the range of about 15 cps to 25 cps is provided, as a solution having a viscosity in the aforementioned range is generally considered optimal for both comfort and retention of the formulation in the eye. The present invention may also include suitable isotonic agents and buffering agents commonly used in ophthalmic formulations may be used, providing that the osmotic pressure of the solution does not deviate from that of lachrymal fluid by more than 2-3% and that the pH of the formulation is maintained in the range of about 6.5 to about 8.0, preferably in the range of about 6.8 to about 7.8, and optimally at a pH of about 7.4. Non-limiting examples of buffering agents include carbonates such as phosphate, sodium and potassium bicarbonate.

The present invention may also be used in a hydrogel, dispersion, or colloidal suspension. Hydrogels are typically made by incorporating a gel-forming polymer such as those set forth above as suitable thickening agents, except that a formulation referred to in the art as a “hydrogel” typically has a higher viscosity than a formulation referred to as a “thickened” solution or suspension. In contrast to such preformed hydrogels, a pharmaceutical composition may also be prepared that forms a hydrogel in situ following application to the eye. Such gels are liquid at room temperature but gel at higher temperatures (and thus are termed “thermoreversible” hydrogels), such as when placed in contact with body fluids. Biocompatible polymers that impart this property include acrylic acid polymers and copolymers, N-isopropylacrylamide derivatives, and block copolymers of ethylene oxide and propylene oxide. The present invention may also be prepared in the form of a dispersion or colloidal suspension. The present invention may also be used in colloidal suspensions formed from microparticles, e.g., microspheres, nanospheres, microcapsules, or nanocapsules, where the microspheres and nanospheres are generally monolithic particles of a polymer matrix in which the pharmaceutical composition is trapped, adsorbed, or otherwise contained, while with microcapsules and nanocapsules, the formulation is actually encapsulated.

Pharmaceutically acceptable ophthalmic carrier(s) for use with the present invention may be of a wide range of types known to those of skill in the art. For example, the present invention can be provided as an ophthalmic solution or suspension, in which case the carrier is at least partially aqueous and can support living cells. The pharmaceutical compositions may also be ointments, in which case the pharmaceutically acceptable carrier comprises an ointment base, e.g., having a melting or softening point close to body temperature, and any ointment bases commonly used in ophthalmic preparations may be advantageously employed. Common ointment bases include petrolatum and mixtures of petrolatum and mineral oil.

As used herein, the term “controlled release” refers to an agent-containing formulation or fraction thereof in which release of the active agent is not immediate, i.e., with a “controlled release” formulation, administration does not result in immediate release of the agent into an absorption pool. The term is used interchangeably with “non-immediate release” as defined in Remington: The Science and Practice of Pharmacy, Nineteenth Ed. (Easton, Pa. Mack Publishing Company, 1995). In general, the term “controlled release” as used herein refers to “sustained release” rather than to “delayed release” formulations. The term “sustained release” (synonymous with “extended release”) is used in its conventional sense to refer to a formulation that provides for gradual release of an active agent over an extended period of time.

In an embodiment, the human corneal epithelial stem cells, the human corneal epithelial stem cell supernatant, or both, and other agents may be released over a period of at least 2, 4, 6, 8, 10, 12 hours, at least 18 hours, at least 24 hours, at least 48 hours, at least 3 days, at least 7 days, or longer. Likewise, the supernatant may be isolated after incubating the cells for at least 18 hours, at least 24 hours, at least 48 hours, at least 3 days, at least 7 days, or longer.

The human corneal epithelial stem cells, the human corneal epithelial stem cell supernatant, or both or pharmaceutical composition can be administered, as described herein, according to any of a number of standard methods including, but not limited to injection, drops, serum, spray, time-release implant, transdermal patch, eye drops, gels, ointments, orally, intraocular injection, subconjuctival injection, peri-/retrobulbar injection, transdermally, or topically to the ocular region by an eye drop dispenser, or the like, including topical intranasal administration or administration by inhalant, and the like, spray, emulsion, suspension, via any drug carriers as sponges, contact lenses, polymers, microspheres, and implants.

A topical administration can be ophthalmic. Topical ophthalmic products may be packaged in multidose form, and may also include preservatives to prevent microbial contamination during use. Suitable preservatives include: biguanides, hydrogen peroxide, hydrogen peroxide producers, benzalkonium chloride, chlorobutanol, benzododecinium bromide, methyl paraben, propyl paraben, phenylethyl alcohol, edetate disodium, sorbic acid, polyquaternium-1, or other agents known to those skilled in the art. Such preservatives are typically employed at a level of from 0.001 to 1% (w/w). Unit dose formulations of the present invention will be sterile, but typically unpreserved. Such formulations, therefore, generally will not contain preservatives.

The pharmaceutical composition may further include antibiotics. Examples of antibiotics include without limitation, cefazolin, cephradine, cefaclor, cephapirin, ceftizoxime, cefoperazone, cefotetan, cefutoxime, cefotaxime, cefadroxil, ceftazidime, cephalexin, cephalothin, cefamandole, cefoxitin, cefonicid, ceforanide, ceftriaxone, cefadroxil, cephradine, cefuroxime, ampicillin, amoxicillin, cyclacillin, ampicillin, penicillin G, penicillin V potassium, piperacillin, oxacillin, bacampicillin, cloxacillin, ticarcillin, azlocillin, carbenicillin, methicillin, nafcillin, erythromycin, tetracycline, doxycycline, minocycline, aztreonam, chloramphenicol, ciprofloxacin hydrochloride, clindamycin, metronidazole, gentamicin, lincomycin, tobramycin, vancomycin, polymyxin B sulfate, colistimethate, colistin, azithromycin, augmentin, sulfamethoxazole, trimethoprim, derivatives thereof, and the like and mixtures thereof.

The pharmaceutical composition may further include corticosteroids. Examples of corticosteroids include cortisone, prednisolone, triamcinolone, flurometholone, dexamethasone, medrysone, loteprednol, fluazacort, hydrocortisone, prednisone triamcinolone, betamethasone, prednisone, methylprednisolone, triamcinolone acetonide, triamcinolone hexacetonide, paramethasone acetate, diflorasone, fluocinolone and fluocinonide, derivatives thereof, and mixtures thereof.

The pharmaceutical composition may further include antihistamines. Examples of antihistamines include, and are not limited to, loradatine, hydroxyzine, diphenhydramine, chlorpheniramine, brompheniramine, cyproheptadine, terfenadine, clemastine, triprolidine, carbinoxamine, diphenylpyraline, phenindamine, azatadine, tripelennamine, dexchlorpheniramine, dexbrompheniramine, methdilazine, and trimprazine doxylamine, pheniramine, pyrilamine, chiorcyclizine, thonzylamine, and derivatives thereof.

As used herein, the terms “effective amount” or “effective doses” refer to that amount of an agent to product the intended pharmacological, therapeutic or preventive results. The pharmacologically effective amount results in the amelioration of one or more signs or symptoms of a disease or condition or the advancement of a disease or conditions, or causes the regression of the disease or condition. For example, if a therapeutically effective amount preferably refers to the amount of a therapeutic agent that decreases the loss of night vision, the loss of overall visual acuity, the loss of visual field, by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or more as compared to an untreated control subject over a defined period of time, e.g., 2 weeks, one month, 2 months, 3 months, 6 months, one year, 2 years, 5 years, or longer. More than one dose may be required to provide an effective dose.

As used herein, the terms “effective” and “effectiveness” includes both pharmacological effectiveness and physiological safety. Pharmacological effectiveness refers to the ability of the treatment to result in a desired biological effect in the patient. Physiological safety refers to the level of toxicity, or other adverse physiological effects at the cellular, organ and/or organism level (often referred to as side-effects) resulting from administration of the treatment. On the other hand, the term “ineffective” indicates that a treatment does not provide sufficient pharmacological effect to be therapeutically useful, even in the absence of deleterious effects, at least in the unstratified population. (Such as treatment may be ineffective in a subgroup that can be identified by the expression profile or profiles.) “Less effective” means that the treatment results in a therapeutically significant lower level of pharmacological effectiveness and/or a therapeutically greater level of adverse physiological effects, e.g., greater liver toxicity.

As used herein, a “subject” refers to living organisms. In certain embodiments, the living organism is an animal, in certain preferred embodiments, the subject is a mammal, in certain embodiments, the subject is a domesticated mammal or a primate including a non-human primate. Examples of subject include humans, monkeys, dogs, cats, mice, rats, cows, horses, goats, and sheep. A human subject may also be referred to as a patient.

As used herein, a subject “suffering from or suspected of suffering from” refers to a specific disease, condition, or syndrome has a sufficient number of risk factors or presents with a sufficient number or combination of signs or symptoms of the disease, condition, or syndrome such that a competent individual would diagnose or suspect that the subject was suffering from the disease, condition or syndrome. As used herein, the specific diseases, conditions, and syndromes are those related to corneal epithelial diseases, including but not limited to: mechanical trauma (e.g. fingernail scratch, contact lens overuse, foreign body in the lid/fornices, trichiasis/distichiasis, chemical exposure); chronic exposure to air (e.g. neurotrophic diseases causing incomplete lid closure such as cranial nerve VII palsy, restrictive eyelid diseases, proptosis, decreased consciousness in drug abuse or comatose state, blepharoplasty, lagophthalmos); ultraviolet burns (e.g. welding, prolonged sun exposure off reflective surfaces); local corneal dryness and systemic disorders leading to corneal dryness (e.g. dry eye syndrome, thyroid eye disease, Sjogren's syndrome, vitamin A deficiency); limbal stem cell deficiency (failure to regenerate epithelial cells, occurs from a variety of causes e.g. chemical burns, post ocular surgery, ocular autoimmune degenerations); topical anesthetic abuse; neurotrophic keratopathy (corneal hypoesthesia or anesthesia caused, most frequently, by damage to the trigeminal nerve, also human simplex virus (HSV), varicella-zoster virus (VZV), and topical drop toxicity, among others), dry eye disease, blepharitis, meibomian gland dysfunction, chronic ocular surface disease, neurotrophic keratoconjunctivitis, corneal ulcer, marginal keratitis, peripheral ulcerative keratitis, acute and chronic keratitis, acute and chronic conjunctivitis, anterior scleritis, corneal abrasion, corneal edema, recurrent corneal erosion, delayed corneal epithelial wound healing, corneal postoperative healing, corneal neovascularization. Subjects suffering from, and suspected of suffering from, a specific disease, condition, or syndrome are not necessarily two distinct groups. Those skilled in the art will realize that other eye diseases, conditions, and syndromes may also benefit from treatment with these corneal epithelial stem cells or their supernatant or both, since all or nearly all eye tissues have a similar origin (i.e., neural crest).

The invention includes formulating ophthalmic compositions, which are microbiologically stable. In some cases, it is possible to formulate preservative-free ophthalmic compositions, which are better tolerable for many patients, in particular patients suffering from an ophthalmic disease.

The following is a more detailed protocol for making and using the cells and supernatants of the present invention. The skilled artisan will understand that certain steps disclosed herein are optional, e.g., the extent to which components are washed, replated, kept in the same plate, etc., without affecting the scope of the present invention. The skilled artisan will understand that under normal circumstances cells may not require as much culture time, while in other cases they require more culture time, depending on the donor (e.g., based on the donor's age) from whom the sample came. The change in time may be 6, 12, 18, 24 hours; or 1, 2, 3, 4, 5, 6, or 7 days.

The present invention may require one of more of the following supplies: Falcon 60×15 mm tissue culture plate, Fisher Scientific, Cat #08-772B; 100×15 mm tissue culture plates, Fisher Scientific, Cat #08-772E; Dissecting Forceps, fine tip autoclaved, VWR, Cat #82027-386; and/or Razor blade, VWR Cat #55411-050. P1000 pipette tips; 15 mL and 50 mL conical tubes; Kim Wipes; 5 mL serological pipets; 10 mL serological pipets; and/or Dropper bottles.

The present invention may require one of more of the following reagents: 70% isopropyl alcohol (IPA); Dulbecco modified eagle medium (DMEM) Media, GIBCO Cat #11885-084; HyClone Standard Fetal Bovine Serum (FBS), heat inactivated, GE Healthcare, Cat #SH30088.03HI; Penicillin-Streptomycin-Glutamine (100×)(PSG), ThermoFisher Scientific Cat #10378016; EpiLife Medium, with 60 μM calcium+supplement ThermoFisher Scientific, Cat #MEPI500CA; Human Corneal Growth Supplement (HCGS), ThermoFisher Scientific, Cat #S-009-5; TrypLE Express Enzyme (1×), ThermoFisher Scientific, Cat #12604021; Minimum Essential Medium, Sigma, Cat #M8042; Phosphate Buffered Saline (PBS) ThermoFisher Scientific, Cat #10010023; and Alcon's Balanced Salt Solution, Company, Item #217103.

The present invention may require one of more of the following pieces of equipment: Biological Safety Cabinet (hood); P1000 pipette; Pipetman; Tissue culture microscope; and a CO₂ incubator, set to 5% CO₂.

Procedure:
1. Make up Media #1 that includes:
   a. DMEM+10% FBS+1% PSG
   b. In hood, mix together the following:
      i. 8.9 mL DMEM media
      ii. 1 mL FBS
      iii. 0.1 mL PSG
   c. Store Media #1 at 4° C. and keep sterile.
2. Mince corneal limbal tissue specimen
   a. Place 2 60×15 mm culture dishes inside hood, one upside down.
   b. Sterilize razorblade with 70% IPA. Wipe off blade with Kim wipe
      i. Be sure blade is completely dry.
   c. Open specimen container and lift tissue out with tweezers. Place onto the inside of the lid of one culture dish. Place the bottom of the dish on the hood surface, with open side down
   d. Add 2 drops of Media #1 to the tissue, just enough to wet it slightly (˜100 μL).
   e. Hold the tissue with the tweezers and mince the tissue vigorously for 2-3 minutes with the razor blade.
   f. Scrape up the minced pieces with the razor blade and tweezers; transfer to the bottom of the culture dish.
   g. Use the tweezers to spread out the minced pieces into an area about the size of a quarter.
   h. Collect any missed pieces from the lid and place in the dish with the other pieces.
   i. Discard the lid used during mincing.
3. Incubate the minced pieces in the hood with no lid for 10-15 minutes for the pieces to dry.
   a. Look for the edges of each piece to be dry. This means they are stuck to the plate.
4. Culture corneal epithelium pieces
   a. Draw up 0.5 mL of Media #1 into a P1000 pipette tip. Dropwise, slowly add media on top of the specimen pieces. Take care not to dislodge the pieces.
      i. Dislodged pieces will never re-attach to the plate and the stem cells inside them will die.
   b. Slowly add another 0.5 mL Media #1 to the dish for a total of 1 mL culture volume
   c. Rotate plate to distribute the media over the whole plate.
      i. This low level of media allows for the specimen pieces to contact both the air and media during culturing.
      ii. Label the lid of the plate with the data and “initial culture”
   d. Check culture dish under the microscope. Look for the following features:
      i. Fibrous tissue—contains no cells.
      ii. Epithelial tissue—these are what is immediately seen. The epithelial cells will die, but their presence helps the stem cells survive somehow.
      iii. Epithelial cells—at the edges of the large chunks of epithelial tissue, a thin layer of epithelial cells is seen. Look for these areas. Stem cells will migrate out at these locations.
      iv. Stem cells are not seen at this time. They migrate out after 1-2 days and attach to the plate.
   e. Incubate 2 days at 37° C., 5% CO₂. Check each day under the microscope
5. Make Media #2
   a. EpiLife Medium+HCGS supplement (no FBS)
   b. In the hood, using sterile technique mix together:
      i. Entire contents of HCGS supplement
      ii. Entire bottle of EpiLife medium.
   c. Store Media #2 at 4° C. and keep sterile.
6. Change media on initial culture
   a. Remove culture dish from incubator; check under microscope
   b. In the hood, tilt plate carefully and remove media from the corner.
   c. Draw up 1 mL of Media #2 and add it slowly, dropwise to the plate.
   d. Rotate the plate to distribute the media.
   e. Place culture dish back in the incubator for 3 days.
   f. Check culture each day under the microscope.
7. Change media every 3 days on initial culture
   a. Remove culture dish from incubator; check under microscope
   b. In the hood, tilt plate carefully and remove media from the corner.
   c. Draw up 1.5 mL of Media #2 and add it slowly, dropwise to the plate.
   d. Rotate the plate to distribute the media.
   e. Place culture dish back in the CO₂ incubator for 3 days.
   f. Check culture each day under the microscope.
8. Pass cells into a 100×15 mm culture plate
   a. Remove culture dish from incubator; check under microscope
   b. In the hood, tilt plate carefully and remove media from the corner.
   c. Add 1.5 mL of TrypLE to the plate; rock plate to distribute then immediately remove and discard.
   d. Add another 1.5 mL of TrypLE to plate and incubate ˜5 minutes in CO₂ incubator.
   e. Visually check the plate. If the cells come off easily with a shake or rotation of the plate, they are ready to be harvested.
   f. Collect the detached cells
      i. Tilt plate
      ii. Draw up the cells in into the pipette tip.
      iii. While the plate is still tilted, eject the liquid at the top of the plate so it washes down to the bottom.
      iv. Repeat 2-3 times
      v. Draw up the cells and transfer to a sterile 15 mL tube
   g. Wash culture plate to collect cells remaining behind
      i. Add 5 mL of Media #2 to the culture plate
      ii. Immediately draw media back up
      iii. Transfer to the same tube containing the cells.
   h. Spin tube at 1200 rpm, 7 minutes, 4° C., break at setting “Level 9”
   i. Decant supernatant
   j. Resuspend cells in 5 mL of Media #2 and add to 100×15 mm culture plate
      i. Label plate with date and passage (“#1”)
   k. Place in CO₂ incubator
   l. Check each day
   m. Change media (5 mL) every 3 days as described above in Step #7
   n. Split (see below) when cells become confluent
9. Split 1 plate into 2 plates
   a. Do this only when cell density is high (confluent, cells cover the whole plate), regardless of when media was changed.
   b. Repeat Step #8 above for TrypLEizing cells, but use 5 mL TrypLE and 10 mL media to wash plate after harvest.
   c. Resuspend cells in 10 mL Media #2 and transfer to 2 100×15 mm culture plates, 5 mL in each plate
      i. Label both plates with the date and passage number (“#2”)
   d. Place in CO₂ incubator
   e. Check each day
   f. Change media (5 mL) every 3 days as described above in Step #7
   g. Split plate when confluent—repeat this step for each plate.
      i. Label each plate with the date and passage number (“#3”, “#4”, etc.)
10. If cells aren't growing well at any point during the culture period
   a. Make up Media #3
      i. Minimal Essential Media (MEM)+20% FBS+1% PSG
      ii. In hood, mix together the following:
         1. 79 mL MEM media
         2. 20 mL FBS
         3. 1 mL PSG
   b. Remove media from plates not growing well
   c. Add corresponding volume of Media #3 to each plate
      i. 1.5 mL for 60×15 mm plates
      ii. 5 mL for 100×15 mm plates
   d. Place in CO₂ incubator
   e. Check each day to monitor health and growth of culture
   f. Change media every 3 days as described above in Step #7 until cultures resume expanding.
   g. Use Media #2 when cultures resume expanding
11. Prepare to harvest Corneal Epithelial Stem Cell supernatant
   a. Do this when 10-12 plates are confluent
   b. Carefully remove media from each plate (tilt plate, draw media up from corner)
   c. Wash each plate 3× with PBS
      i. Gently add 2 mL PBS to plate; rock to distribute
      ii. Tilt plate and remove PBS
      iii. Repeat 2 more times for 3× wash
   d. Add 4-5 mL of PBS to each plate and incubate overnight in CO₂ incubator
12. Harvest supernatant
   a. Remove PBS from each plate and pool into a single 50 mL conical tube
   b. Spin tube at 1200 rpm, 7 min, 4° C., break at setting “Level 9”
   c. Transfer supernatant into fresh 50 mL tube.
   d. Fill the dropper bottles provided by Dr. Rush
   e. Label dropper bottles as described below:
13. Prepare droppers for shipping

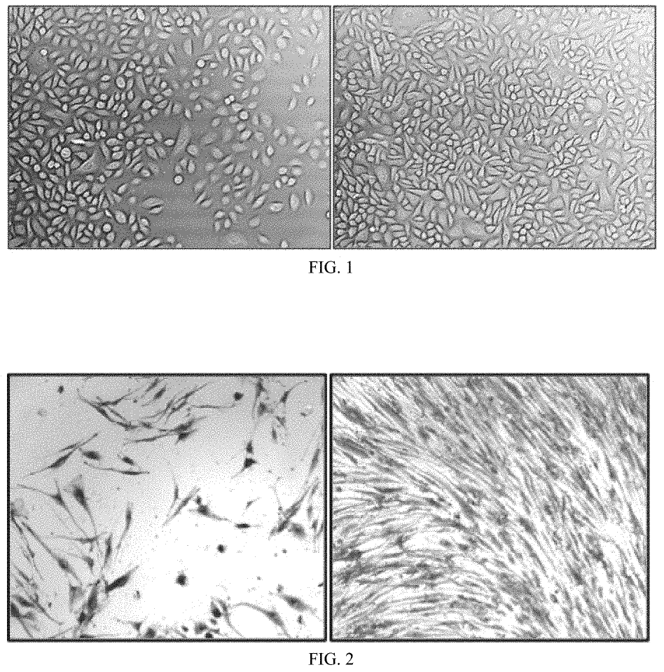

FIG. 1 is a micrograph of an image of short-term survival (variant of) corneal epithelial stem cells, showing their expansion over 10 to 20 days. FIG. 2 is a micrograph of an image of long-term survival (variant of) corneal epithelial stem cells, showing their expansion over 3 to 6 months.

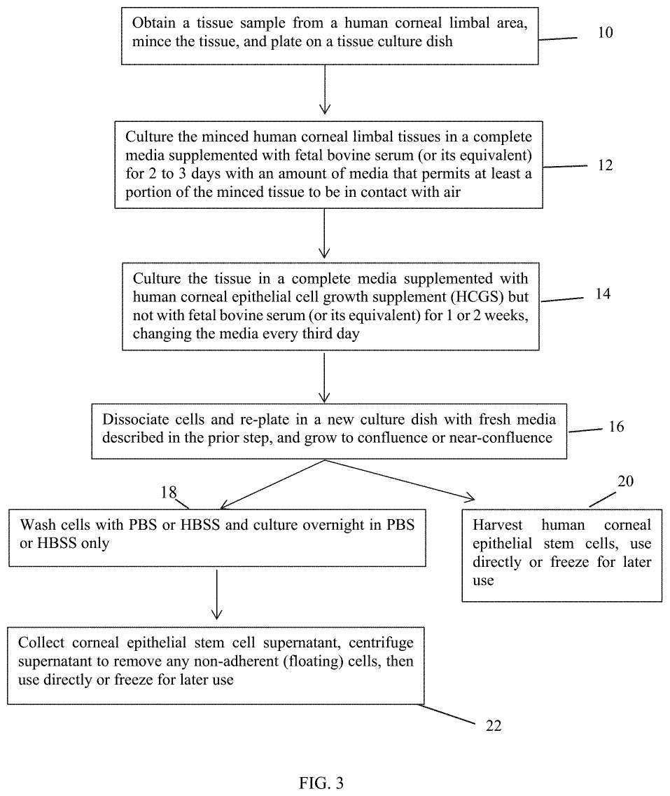

FIG. 3 is a flow chart of one embodiment of one method of the present invention. To generate human corneal epithelial stem cells, a human corneal epithelial stem cell supernatant, or both, the method comprises (not showing Step #s 1 & 5): (Step #2) obtain a tissue sample from a human corneal limbal area, mince the tissue, and plate on a tissue culture dish; (Step #s 3&4) culture the minced human corneal limbal tissues in cell culture media supplemented with fetal bovine serum (or its equivalent) for 2 to 3 days with an amount of media that permits at least a portion of the minced tissue to be in contact with air; (Step #s 6&7) culture the tissue in corneal epithelial cell culture media with human corneal epithelial cell growth supplement (HCGS) without fetal bovine serum or its equivalent (i.e., media from Step #5) for 1 to 3 weeks, changing the media every third day; and (Step #8) dissociate cells and re-plate in a new culture dish with fresh media from Step #5, and grow to confluence or near-confluence. At this time, the cells can be harvested directly, or (Step #9 and Step #s 11 &12) recultured to obtain additional cells, supernatant or both. The corneal epithelial stem cell culture supernatant can be made by: (Step #11) washing the cells with PBS or HBSS and culturing overnight in PBS or HBSS only; and (Step #12) collecting the corneal epithelial stem cell supernatant and centrifuging the supernatant to remove any non-adherent (floating) cells. The supernatant can then be used directly or frozen for later use, as in Step #13.

In one embodiment, the present invention includes a method of generating a population of human corneal epithelial stem cells consists essentially of, or consists of: wetting and mincing a corneal epithelial sample in a media; drying the minced corneal epithelial sample until sample edges are adhered to a substrate; adding a growth media comprising fetal bovine serum or human serum to the minced corneal epithelial sample without dislodging the minced corneal epithelial sample from the substrate with an amount of media that permits at least a portion of the minced corneal epithelial sample to be in contact with air; culturing the minced corneal epithelial sample for one or more days; changing the growth media to a media comprising a human corneal growth supplement (HCGS) with no fetal bovine serum; culturing the cells for 1 to 3 weeks; and harvesting the human corneal epithelial stem cells. In one aspect, the method further comprises: dissociating a population of human corneal epithelial cells isolated to generate a population of dissociated human corneal epithelial cells; culturing the dissociated human corneal epithelial cells in a media comprising fetal bovine serum or human serum until the cells grow (typically 1 to 3 weeks).

Topical Cadaver-Derived Corneal Epithelial Stem Cell Products in the Treatment of Severe Dry Eye Disease.

The present invention includes a novel transplantable, self-administered topical cadaver-derived corneal epithelial stem cell product for treatment of severe dry eye disease (DED). Thirty four eyes of 17 patients with advanced DED as defined by Standardized Patient Evaluation of Eye Dryness (SPEED™) questionnaire ≥14, Ocular Surface Disease Index (OSDI©) score ≥40 and documented attempt of at least six conventional dry eye therapies were enrolled into a prospective clinical trial at a single private practice institution. Treatment consisted of patient self-administered topical instillation of the corneal epithelial stem cell-derived product four times daily in both eyes for a total of 12 weeks. Patient reported outcome measures (PROMs) were taken with the SPEED™ questionnaire (the main outcome variable), OSDI© score and visual analog score (VAS; UNC Dry Eye Management Scale©), and objective clinical measurements were taken with best-corrected visual acuity (BCVA), corneal topographic index measurements and tear film osmolarity. These measurements were compared at baseline versus the time period at the completion of the 12 week treatment.

Results: All 34 eyes tolerated the treatment well without any notable adverse events, significant side effects or other complications associated with the treatment. Compared with baseline, both the SPEED™ questionnaire and the VAS significantly improved at the conclusion of the 12 week treatment (p=0.0054 and p=0.0202, respectively). The OSDI© improved by an average of 10.9 points after the treatment but was not statistically significant (p=0.1409). There were no significant changes in any of the objective clinical measurements. None of the study subjects failed to complete the treatment course, experienced decrease in any of the PROMs or lost one or more lines of BCVA during the follow-up period.

It was found that topical corneal epithelial stem cell-derived products that can be self-administered by the patient provide improving patient symptoms and quality of life in the setting of severe DED that is poorly controlled or unresponsive to conventional therapies.

The prevalence of dry eye disease (DED) has been estimated to be as high as 50% of the population.¹ Systematic literature reviews have detailed the substantial economic liability of DED including the loss in work productivity.²⁻³ The overall annual burden of DED on the United States healthcare system and society at large may well be in excess of $50 billion (USD).⁴ While there is a definite predilection for DED in females and in patients with autoimmune disorders, DED occurs in all ethnicities and in all population demographics as age increases.⁵⁻⁸ The treatment strategy for DED may vary depending upon the underlying etiology of the dry eye (aqueous deficiency versus evaporative), clinical examination findings and the presence of other associated ocular surface diseases.⁹ There are many conventional therapies for DED which include ocular lubricants, oral essential fatty acid supplementation, lid hygiene and warm compresses, punctal occlusion, various treatments to obstructed meibomian glands, topical antibiotics, topical corticosteroids, topical secretagogues, topical non-glucocorticoid immunomodulatory drugs and scleral contact lenses.¹⁰⁻¹¹ Even with the current multitude of therapeutic options, investigators have observed the urgent need to develop more safe and effective treatment modalities.¹² Furthermore, evaluating response to treatment for DED has always been challenging due to the fact that there is a poor correlation among the patient's symptoms and the objective clinical findings.¹³⁻¹⁴

Knowing that there is tremendous need for improved therapeutic options, there has been considerable effort to elucidate the underlying pathophysiological mechanisms of DED at both the cellular and molecular levels. Several recent studies have described in great detail the biologic niche for both the corneal limbal epithelial stem cells and the conjunctival goblet cells.¹⁵⁻¹⁶ These ocular surface epithelial cells secrete mucins that form a hydrophilic barrier for the protection and lubrication of the eye.¹⁷⁻¹⁹ This complex interaction of proteins in the extracellular matrix consists of glycosylated membrane-associated mucins that contain lattices of galectin-3 and other integrated proteins to form the glycocalyx structure.²⁰⁻²¹ It has been hypothesized that alteration and dysfunction in mucin-associated homeostasis is a major contributor in the pathogenesis of DED.²²⁻²⁵ This knowledge has been applied to the development of more innovative treatments for ocular surface regeneration which include biologic agents such as growth factors, blood products and cell-based therapies.²⁶⁻²⁷ For example, autologous serum eye drops, which contain biochemical components that more closely mimic natural tears, have shown superiority relative to plain lubricating eye drops in the treatment of chronic ocular surface diseases, and it is gaining more widespread acceptance and use in the treatment of more advanced cases of DED.²⁸ With reasonable degree of success, investigators have also tried other biologic agents that are not naturally indigenous to the ocular surface such as amniotic membrane grafts that are derived from a donated mother's placenta to help promote ocular surface healing.²⁹ The only regenerative biologic treatment that is native and specific to the eye requires staged culturing of autologous or allogenic corneal epithelial stem cells for several weeks followed by surgical transplantation of the newly created graft onto the ocular surface, a treatment that has been used primarily in the setting of limbal stem cell deficiency and not DED.³⁰⁻³²

Presently there are no reports describing a safe and effective therapeutic biologic agent for DED that does not either require invasive surgery or require biologic material that is not specific and differentiated for the ocular surface. The results herein are the first-in-human use of a novel, patient-delivered topical application of a corneal epithelial stem cell-derived product for the treatment of severe DED.

Study Design. The Salus Independent Review Board (IORG0005674) approved this prospective pilot case series of severe DED patients that underwent topical, self-administered treatment with the corneal epithelial stem cell-derived product from May 2019 through December 2019 at a single private practice institution in Amarillo, Tex., USA. All components of the study adhered to the tenets of the Declaration of Helsinki and were performed in accordance with human research standards and regulations. The study is registered at ClinicalTrials.gov (NCT03302273, last accessed Jan. 1, 2020).

Participants. Consecutive patients with severe DED presenting to a single clinical practice were assessed for study eligibility. Inclusion criteria was comprised of clinical diagnosis of dry eye syndrome, severe DED (as defined by Standardized Patient Evaluation of Eye Dryness (SPEED™) questionnaire ≥14, Ocular Surface Disease Index (OSDI©) score ≥40, and documented attempt and/or current use of at least six conventional dry eye therapies¹⁰⁻¹¹), age 25-75, and willingness and ability to participate in a research trial. Exclusion criteria consisted of inability or unwillingness to participate in an investigational study. Both eyes in all study subjects were treated simultaneously according to the protocol. All enrolled subjects were given a written informed consent.

Randomization and Masking. There was no active comparator in the study since it was a pilot series. The study participants were not aware of the study design and intent. Only the ophthalmic technicians that collected the objective study data (see below) were masked as to which patients were enrolled into the research trial. The treating physician (SWR) was an unmasked observer.

Intervention. Enrolled study subjects that signed the written informed consent document were given instructions on the treatment plan. The treatment consisted of patient-administered topical instillation of the corneal epithelial stem cell-derived eye drop product (described below) four times daily (QID) to both eyes for a total of 12 weeks. The patient was instructed to refrigerate the stem cell-derived product which was dispensed in eye-dropper bottles in 5 mL aliquots. Patients continued treatment as usual with all prior therapies. No changes were made to any of these existing dry eye treatments during the study interval.

Harvesting of the Corneal Epithelial Stem Cells. The corneal epithelial stem cells were derived from corneoscleral rim cadaver donors. All corneal donors were transplantable grade tissue that were received from an Eye Bank Association of America (EBAA) accredited facility and had negative serology testing. The harvesting technique was initiated under sterile environment where the anterior sections of the tissue are trimmed to contain the epithelial side of sclera, conjunctiva and cornea. This tissue was delivered to the manufacturing laboratory at the participating blood bank (see below). The tissue was transported in 10 mL of corneal preservation media, Optisol-GS (minimal essential media (MEM)/TC-199 media supplemented with 2.5% chondroitin sulfate, 1% dextran 40, 0.1 mmol/L nonessential amino acids, 1 mmol/L sodium pyruvate, 181 mEq/L sodium ion, and undisclosed amounts of HEPES buffer, gentamicin sulfate, sodium bicarbonate, and additional antioxidants).

Culturing and Expansion of the Corneal Epithelial Stem Cells. The harvested corneal epithelial stem cells were transferred to a licensed blood and tissue facility (Oklahoma Blood Institute, 901 N. Lincoln Blvd, Oklahoma City, Okla. 73104, USA). The facility is accredited by the American Association of Blood Banks (AABB) and compliant with all applicable registration and regulatory requirements for the handling and manufacturing of Human Cells, Tissues, and Cellular and Tissue-Based Products (HCT/Ps). The tissue was processed using sterile forceps and a razorblade to mince it into 12-15 small pieces (each 1-3 mm long) on the lid of a Petri dish. The tissue pieces were spread onto the bottom of a 60 mm Petri dish and allowed to dry and adhere to the dish for approximately 15 minutes. One mL of MEM supplemented with 10% human serum and 1% penicillin/streptomycin/glutamate was added to the dish and the tissue pieces were incubated at 37° C., 5% CO₂ for 2 days. Next, the media was removed from the dish, replaced with 1.5 mL of serum-free, antibiotic-free EpiLife media with calcium (ThermoFisher Scientific) containing Human Corneal Growth Supplement (HCGS, ThermoFisher Scientific), and the dish was incubated at 37° C., 5% CO₂. EpiLife/HCGS media was exchanged every 3 days and cultures were monitored for the emergence of corneal epithelial and limbal stem cells from the tissue pieces onto the plastic dish surface. Tissue pieces were sterilely removed from the culture 7-21 days after culture initiation and transferred to a new dish to start a new culture. Cells adherent to the dish surface were left to expand for 14-28 days after culture initiation. When cells reached 80% confluency in the initial Petri dish, they were removed with TripLE Express (ThermoFisher Scientific), washed, and used to reseed 1 T-75 culture flask containing 7.0 mL of EpiLife/HCGS media. The culture was incubated at 37° C., 5% CO₂, fed every 3 days by media exchange, and monitored for cell growth. When the culture reached 80% confluency in the T-75 flask, the cells were removed with TripLE Express, washed, and used to reseed 1 T-150 flask containing 14.0 mL of EpiLife/HCGS media. The culture was incubated at 37° C., 5% CO₂, fed every 3 days by media exchange, and monitored for cell growth. When the culture reached 90-100% confluency in the T-150 flask, media was removed, and the flask was washed 2 times with 10 mL phosphate buffered saline (PBS).

Manufacturing of the Final Corneal Epithelial Stem Cell Product. The final product was prepared according to Current Good Manufacturing Practice (cGMP) requirements and with minimal manipulation and a clinical application for homologous use according the FDA's current thinking and guidance for industry regarding tissue-based products.³³ To manufacture the treatment supplement, 19 mL of PBS was added to the flask and incubated for 28-32 hours at 37° C., 5% CO₂. The supernatant was then collected, diluted with 19 mL of PBS, and centrifuged at 1500 RPM for 10 minutes to pellet any cells contaminating the supernatant. The diluted acellular supernatant was transferred to 6 sterile eye dropper bottles, ˜6.0 mL/bottle. A small volume of supernatant was reserved for sterility testing (BacT/ALERT). The dropper bottles were sealed, labeled, and stored at −15° C. to −25° C. until the results of sterility testing were complete. Supernatant lots with negative 14-day sterility testing results were released for use in treatment.

Data Collection, Assessments and Outcome Measures. The demographic and baseline characteristics collected from each study participant included age, gender, ethnicity, type and number of previous and current conventional dry eye treatments used, associated autoimmune disorders, other systemic and ocular co-morbidities, and lens status. Subjective patient reported outcome measures (PROMs) were taken with the SPEED™ questionnaire³⁴⁻³⁵ (the main outcome variable), OSDI© score³⁶ and visual analog score (VAS; UNC Dry Eye Management Scale).³⁷⁻³⁸ Objective measurements included best-corrected visual acuity (BCVA), tear film osmolarity and corneal topographic measurements (surface regularity index (SRI), projected visual acuity (PVA) and surface asymmetry index (SAT)) using the TMS-4 Topographer (Tomey; Phoenix, Ariz., USA). The objective measurements were averages among both eyes for each study patient. All outcome variables were taken at baseline (4 weeks prior to treatment and then again immediately prior to treatment) and at 4 weeks, 8 weeks, 12 weeks after treatment initiated and then finally at 12 weeks after the treatment course was completed.

Sample Size, Power Calculation and Statistical Analysis. Standard deviation of the main outcome variable (SPEED™ questionnaire) was determined to be 8 by a pre-treatment sampling of the first four enrolled patients. Using power of 90%, alpha of 0.05 and difference to detect of 8 (33% difference from the sampling mean), the sample size was calculated to be 13 patients. The JMP 11 software from the SAS Institute (Cary, N.C., USA) was used to analyze distributions and calculate means with standard deviations. One-way analysis of the variance was used to compare means of the baseline measurements versus the post-treatment measurements. Visual acuity change was considered significant if there was change by log MAR 0.3 or more, whereas the other comparisons were considered statistically significant at the p<0.05 level.

Safety Monitoring. The enrolled study patients were assessed at 2 weeks after initiation of treatment in addition to each scheduled data collection appointment for symptoms and clinical findings of side effects or adverse events. Patients were given 24 hour emergency contact information for any concerns. Any suspected or known adverse events were immediately reported to the Salus Independent Review Board according to approved study protocol guidelines.

Results. A total of 22 consecutive patients with severe DED met eligibility criteria during the enrollment period and were presented with the opportunity to participate in the clinical trial. Five of these patients (22.7%) either declined or were unable to enroll into the study. Therefore, there were 34 eyes of 17 patients included in the analysis, all of whom completed the study (100% completion rate). A flow chart of the study enrollment data is given in FIG. 3. All patients reported treatment compliance and completion of the 12 week treatment. One patient had a 3 week interruption in the treatment but still completed a 12 week course. The baseline characteristics and demographic features of the study population are summarized in Table 1. The average number of previous DED treatments that failed to achieve symptom stability for the study population was 9.1 (+/−2.6), but no specific algorithm had been used to determine the types, order of use, or combinations of treatments that were used for any particular patient. Furthermore, all of the patients had previous care delivered by multiple providers. None of the patients with history of LASIK had the surgery done within the past 3 years.

TABLE 1
Corneal Epithelial Stem Cell-Derived Therapy for Dry Eye Disease.
Baseline Demographics and Characteristics of the Study Population.

| Baseline Characteristics and | |
|---|---|
| Demographics (n = 34 | |
| eyes of 17 patients) | Means with (Standard Deviations) |
| Age (years) | 57.9 (+/−13.7) |
| | Range = 27 to 75 |
| Gender | Male = 0 (0.0%) |
| | Female = 17 (100.0%) |
| Ethnicity | Caucasian = 16 (94.1%) |
| | Hispanic = 1 (5.9%) |
| | Other = 0 (0.0%) |
| Number of Previous Dry | 9.1 (+/−2.6) |
| Eye Treatments | Range = 6 to 14 |
| Treatment Distributions | Artificial Tears = 17 (100.0%) |
| | Lubricating Ophthalmic |
| | Ointment = 17 (100.0%) |
| | Topical Cyclosporine = 17 (100%) |
| | Topical Corticosteroids = 16 (94.1%) |
| | Punctal Occlusion = 13 (76.5%) |
| | Oral Omega-3 |
| | Supplement = 12 (70.6%) |
| | Warm Compresses and |
| | Lid Scrubs = 10 (58.8%) |
| | Moisture Chamber Goggles, |
| | Sleep Masks or other |
| | Eyewear Products = 7 (41.2%) |
| | Topical Lifitegrast = 6 (35.3%) |
| | Autologous Serum/Blood |
| | Products = 6 (35.3%) |
| | Hydroxypropyl Cellulose |
| | Ophthalmic Inserts = 6 (35.3%) |
| | Eyelid Thermal Pulsation = 5 (29.4%) |
| | Other Anti-inflammatory |
| | Systemics = 5 (29.4%) |
| | Systemic Cholinergic |
| | Agonists = 3 (17.6%) |
| | Amniotic Membrane |
| | Grafting = 2 (11.8%) |
| | Meibomian Gland |
| | Expression = 2 (11.8%) |
| | Topical Antihistamines = 2 (11.8%) |
| | Topical Antibiotics = 2 (11.8%) |
| | Scleral Contact Lenses = 1 (5.9%) |
| | Liposome Spray = 1 (5.9%) |
| | Topical Chondroitin |
| | Sulfate = 1 (5.9%) |
| Previous Use of Autologous | Yes = 6 (35.3%) |
| Serum or other Topical | No = 11(64.7%) |
| Blood-derived Product | |
| Autoimmune Disorder Diagnosis | Yes = 7 (41.2%)* |
| | No = 10 (58.8%) |
| | Sjogren's Syndrome = 6 |
| | Rheumatoid Arthritis = 2 |
| | Systemic Lupus Erythematosus = 2 |
| | Scleroderma = 1 |
| | (Some study subjects had |
| | multiple diagnoses)* |
| Other Systemic Disorders | Yes = 6 (35.3%) |
| | No = 11(64.7%) |
| | Thyroid Disorder = 4 |
| | Atopy = 2 |
| | Diabetes Mellitus = 1 |
| | Graft versus Host |
| | Disease (GVHD) = 1 |
| Other Ocular Comorbidities | Yes = 3 (17.6%) |
| | No = 14 (82.4%) |
| | Herpes Zoster Ophthalmicus = 1 |
| | Floppy Eyelid Syndrome = 1 |
| | Superior Limbic Keratitis = 1 |
| | Salzmann Nodular Degeneration = 1 |
| History of Previous | Yes = 4 (LASIK = 4, PRK = 0 |
| Refractive Surgery | and RK = 0) (23.5%) |
| | No = 13 (76.5%) |
| Lens Status | Phakic = 12 (70.6%) |
| | Pseudophakic = 5 (29.4%) |

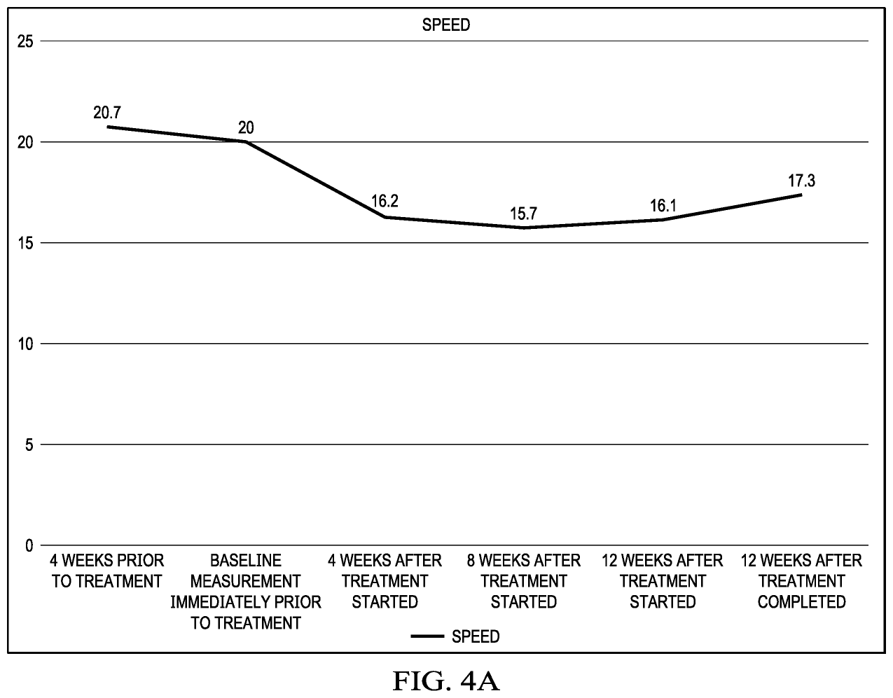

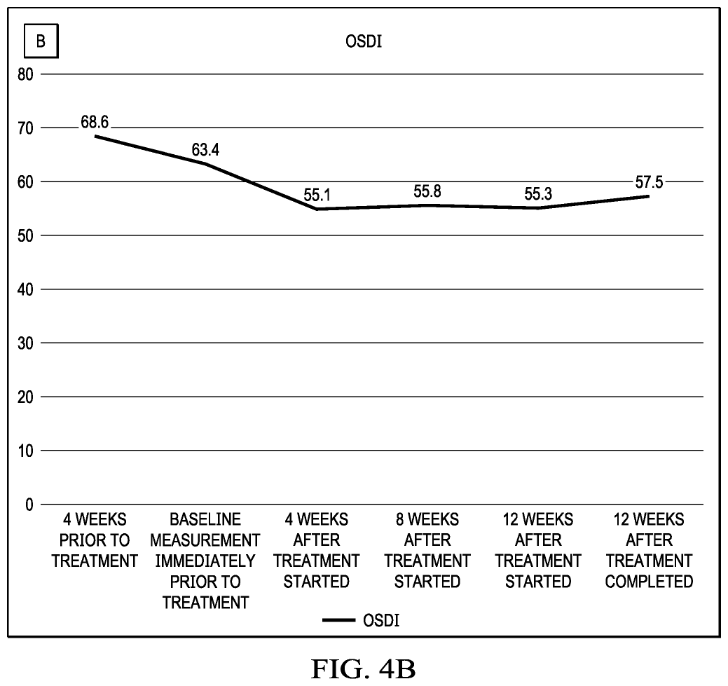

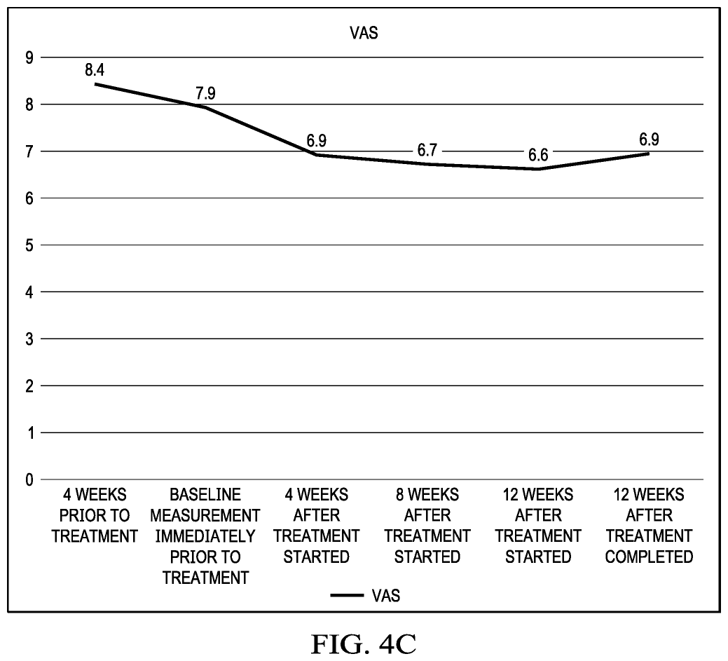

The outcome comparison among average baseline versus 12 week post-treatment outcomes are detailed in Table 2. The main outcome variable (SPEED™ questionnaire) significantly improved from baseline by an average of 4.7 points (23.0%) (p=0.0054). OSDI© score improved from baseline by an average of 10.9 points (17.1%) (p=0.1409, not statistically significant) and VAS improved from baseline by an average of 1.1 points (14.1%) (p=0.0202). FIGS. 4A to 4C show the trend in all three of the measured PROMs over the treatment course. No patients had worsening in any of the PROMs throughout the duration of the follow-up time interval during the treatment. BCVA and tear film osmolarity showed a trend for improvement but did not achieve statistical significance (p=0.5678 and p=0.1884, respectively). None of the other objective clinical measurements showed significant changes.

FIG. 4A to 4C shows the results from corneal epithelial stem cell-derived therapy for dry eye disease. Mean Patient Reported Outcome Measurements of over Various Time Intervals during the 12 Week Treatment Period for (FIG. 4A) Standardized Patient Evaluation of Eye Dryness (SPEED™) Questionnaire, (FIG. 4B) Ocular Surface Disease Index (OSDI) Score and (FIG. 4C) Visual Analog Scale (VAS).

TABLE 2
Corneal Epithelial Stem Cell-Derived Therapy for
Dry Eye Disease. Average Baseline versus
Twelve Week Post-Treatment Outcome Comparisons.

| | Average | Post-Treatment | |
|---|---|---|---|
| | Baseline Values | Values | |
| Outcomes | Means with (95% | Means with (95% | |
| (n = 34 eyes | Confidence | Confidence | p- |
| of 17 patients) | Intervals) | Intervals) | value |
| Standardized Patient | 20.4 (18.1-22.7) | 15.7 (13.4-18.0) | 0.0054 |
| Evaluation of | | | |
| Eye Dryness | | | |
| (SPEED ™) | | | |
| Questionnaire | | | |
| (scaled 0 to 28 with | | | |
| 28 being the worst) | | | |
| Ocular Surface | 63.4 (53.2-73.6) | 52.5 (42.0-63.1) | 0.1409 |
| Disease Index | | | |
| (OSDI ®) Score | | | |
| (scaled 0 to 100 | | | |
| with 100 | | | |
| being the worst) | | | |
| Visual Analog | 8.0 (7.3-8.6) | 6.8 (6.2-7.5) | 0.0202 |
| Scale (VAS) | | | |
| (scaled 1 to 10 | | | |
| with 10 being the | | | |
| worst) | | | |
| Best Spectacle | 0.18 (0.15-0.25) | 0.15 (0.07-0.22) | 0.5678 |
| Corrected Visual | | | |
| Acuity (logMAR) | | | |
| Tear Film Osmolarity | 318.9 (306.6-331.1) | 307.3 (294.6-320.0) | 0.1884 |
| (mOSM/L) | | | |
| Topographic Surface | 0.70 (0.51-0.89) | 0.75 (0.55-0.95) | 0.7413 |
| Regularity | | | |
| Index (SRI) | | | |
| Topographic | 0.12 (0.08-0.17) | 0.13 (0.08-0.18) | 0.8673 |
| Projected Visual | | | |
| Acuity (PVA) | | | |
| (logMAR) | | | |
| Average | 0.81 (0.57-1.05) | 1.02 (0.77-1.26) | 0.2240 |
| Topographic Surface | | | |
| Asymmetry | | | |
| Index (SAI) | | | |

Change in the four week pre-treatment baseline from immediate pre-treatment baseline PROMs was compared to change in immediate pre-treatment baseline from average post-treatment PROMs at 12 weeks. All three outcomes showed significant improvement using this comparison technique (Table 3). In addition, extended follow-up comparison was done with the time point 12 weeks after the treatment was completed (24 weeks from baseline at the time treatment was originally started) which showed a small upward trend to baseline but was not statistically significant.

TABLE 3
Corneal Epithelial Stem Cell-Derived Therapy
for Dry Eye Disease. Patient Reported
Outcome Measurement Comparisons for Change in Four
Week Pre-Treatment Baseline from Immediate Pre-Treatment
Baseline versus Change in Immediate Baseline from Average Post
Treatment Values.

| | Change in Four | Change in | |
|---|---|---|---|
| | Week Pre- | Immediate Pre- | |
| | Treatment | Treatment | |
| | Baseline from | Baseline from | |
| | Immediate | Average | |
| | Pre-Treatment | Post-Treatment | |
| | Baseline Values | Values | |
| Outcomes | Means with (95% | Means with (95% | |
| (n = 34 eyes | Confidence | Confidence | p- |
| of 17 patients) | Intervals) | Intervals) | value |
| Standardized Patient | +0.7 | −4.4 | 0.0077 |
| Evaluation of Eye Dryness | (−2.1 to +3.4) | (−6.6 to −2.1) | |
| (SPEED ™) Questionnaire | | | |
| Ocular Surface Disease | −0.2 | −15.7 | 0.0110 |
| Index (OSDI ®) Score | (−8.9 to +8.5) | (−23.3 to −8.2) | |
| Visual Analog Scale (VAS) | −0.15 | −0.98 | 0.0372 |
| | (−0.75 to +0.45) | (−1.48 to −0.49) | |

Subset analysis was done to determine if there were any significant differences in response to therapy among patients that have an underlying autoimmune disorder or among patients that have previously tried autologous serum (or other topical blood-derived products). In both instances, the patients in these categories responded similarly to those in the remaining cohort without any apparent statistical trends.

Exit surveys were given to get additional patient feedback. All 17 patients stated that the study treatment was better than artificial tears and that they would want to do it again. All patients also reported decreased use of artificial tears PRN during the treatment period. Fifteen patients (88.2%) described the treatment as “soothing” when they used the eye drops. All 17 of the patients in this study were either currently using or had previously used cyclosporine ophthalmic emulsion (Restasis®), and 100% of them stated that the treatment used in this clinical trial was preferred and even superior with regards to alleviating their dry eye symptoms over a 12 week period.

There were no identifiable trends for severe side effects or adverse events during the study period for any of the enrolled study participants. Lengthy questionnaire with ocular symptoms were administered to each patient. With regard to subjective symptoms, 2 patients noticed transient stinging/burning, 2 patients noticed mild aftertaste, 1 patient noticed itching, and 1 patient noticed mattering/crusting that correlated with instillation of the drops. As it relates to objective clinical findings, the examiner noticed subconjunctival hemorrhage in one patient over the duration of the study interval. There were no other observable examination findings the correlated with use of the therapy.

This is the first human clinical trial demonstrating effective use of a corneal epithelial stem cell-derived biologic agent in the setting of DED. Furthermore, lack of noticeable side effects and adverse events demonstrate its preliminary safety as a viable treatment for DED. There is a growing body of literature regarding biologic treatments for DED that will promote ocular surface regeneration. Compared to other experimental biologic treatments, the topical administration of this product by the patient obviates the need for surgical intervention as is the case for limbal epithelial transplants.³⁰⁻³² Another advantage of this treatment over autologous serum and other non-allogenic blood-based products is that there is no need for frequent blood draws or finger pricks. With particular regards to autologous serum eye drops, systematic reviews have shown their failure to improve PROMs in the setting of DED.³⁹

Since it is well-known that treatment satisfaction in the setting of DED is underestimated and does necessarily not correlate with objective clinical outcome measures,⁴⁰ more investigators are starting to rely exclusively on the impact on quality of life and the PROMs in order to gauge the response to treatment.⁴¹ Even with classic clinical measures such as Schirmer's testing, tear break up times and corneal staining findings along with newer diagnostic technology using measurements of tear film osmolarity, matrix metalloproteinase 9 and various ocular surface/meibomian gland imaging modalities, authors have concluded that there still exists no gold standard for the diagnosis and monitoring of DED.⁴² For this reason the future development of biologic agents in the treatment of DED must rely more upon demonstrating improvement in PROMs in order to translate into improved quality of life. Demonstrating statistically significant improvement in two of the three PROMs evaluated for a DED treatment in the biological realm is a relative strength of this study. The OSDI© score, BCVA and tear osmolarity all started to show a trend for improvement, but the study was not adequately powered to determine statistical significance.

In this study, a subset analysis of patients with previous failed use or current use of autologous serum (n=6) showed no significant difference in response to therapy as measured by change in SPEED™ questionnaire compared to those patients that have never tried autologous serum previously (p>0.05). By way of explanation, and in no way a limitation of the present invention, a non-homogenous biologic product may have a different therapeutic mechanism of action in the treatment of DED than blood-based biologic products. The composition of this eye-specific product is distinct from other non-homogenous blood-based biologic products in that laboratory testing has shown that it contains galectin-3 and other glycocalyx components that will serve as a protective barrier to the diseased ocular surface.

This study included exclusively females. The gender disparity for DED has been notable in prior studies.⁵ In clinical practice it is unusual to encounter males with ^{OSDI}© score greater than 40. A study that had recruited patients with all severity levels of DED would have equalized the gender imbalance to some degree.

In summary, the present invention is a novel and transplantable corneal epithelial stem cell-derived product that is comprised of supernatant containing glycocalyx components that can be self-administered by the patient. The treatment on severe DED patients demonstrated significant outcomes with regards to efficacy for the improvement of PROMs specific for DED.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or human serum, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

U.S. Pat. No. 5,585,265

U.S. Pat. No. 5,672,498

U.S. Pat. No. 5,786,201

U.S. Pat. No. 6,984,622

U.S. Pat. No. 9,574,171

U.S. Patent Application No. US20020039788A1

U.S. Patent Application No. US20050186672A1

1. Craig J P, Nelson J D, Azar D T, Belmonte C, Bron A J, Chauhan S K, de Paiva C S, Gomes J A P, Hammitt K M, Jones L, Nichols J J, Nichols K K, Novack G D, Stapleton F J, Willcox M D P, Wolffsohn J S, Sullivan D A. TFOS DEWS II Report Executive Summary. Ocul Surf. 2017 October; 15(4): 802-812.

2. McDonald M, Patel D A, Keith M S, Snedecor S J. Economic and Humanistic Burden of Dry Eye Disease in Europe, North America, and Asia: A Systematic Literature Review. Ocul Surf. 2016 April; 14(2): 144-67.

3. Pflugfelder S C. Prevalence, burden, and pharmacoeconomics of dry eye disease. Am J Manag Care. 2008 April; 14(3 Suppl): S102-6.

4. Yu J, Asche C V, Fairchild C J. The economic burden of dry eye disease in the United States: a decision tree analysis. Cornea. 2011 April; 30(4): 379-87.

5. Sullivan D A, Rocha E M, Aragona P, Clayton J A, Ding J, Golebiowski B, Hampel U, McDermott A M, Schaumberg D A, Srinivasan S, Versura P, Willcox MDP. TFOS DEWS II Sex, Gender, and Hormones Report. Ocul Surf. 2017 July; 15(3): 284-333.

6. Chen X, Wu H, Wei W. Advances in the diagnosis and treatment of Sjogren's syndrome. Clin Rheumatol. 2018 July; 37(7): 1743-1749.

7. Fox R I. Sjögren's syndrome. Lancet. 2005 Jul. 23-29; 366(9482): 321-31.

8. Stapleton F, Alves M, Bunya V Y, Jalbert I, Lekhanont K, Malet F, Na K S, Schaumberg D, Uchino M, Vehof J, Viso E, Vitale S, Jones L. TFOS DEWS II Epidemiology Report. Ocul Surf 2017 July; 15(3): 334-365.

9. Craig J P, Nichols K K, Akpek E K, Caffery B, Dua H S, Joo C K, Liu Z, Nelson J D, Nichols J J, Tsubota K, Stapleton F. TFOS DEWS II Definition and Classification Report. Ocul Surf 2017 July; 15(3): 276-283.

10. Jones L, Downie L E, Korb D, Benitez-Del-Castillo J M, Dana R, Deng S X, Dong P N, Geerling G, Hida R Y, Liu Y, Seo K Y, Tauber J, Wakamatsu T H, Xu J, Wolffsohn J S, Craig J P. TFOS DEWS II Management and Therapy Report. Ocul Surf 2017 July; 15(3): 575-628.

11. Thulasi P, Djalilian A R. Update in Current Diagnostics and Therapeutics of Dry Eye Disease. Ophthalmology. 2017 November; 124(11S): S27-S33.

12. Al-Saedi Z, Zimmerman A, Bachu R D, Dey S, Shah Z, Baugh R, Boddu S H. Dry Eye Disease: Present Challenges in the Management and Future Trends. Curr Pharm Des. 2016; 22(28): 4470-90.

13. Kyei S, Dzasimatu S K, Asiedu K, Ayerakwah P A. Association between dry eye symptoms and signs. J Curr Ophthalmol. 2018 Jun. 28; 30(4): 321-325.

14. Bron A J, Tomlinson A, Foulks G N, Pepose J S, Baudouin C, Geerling G, Nichols K K, Lemp M A. Rethinking dry eye disease: a perspective on clinical implications. Ocul Surf 2014 April; 12(2 Suppl): S1-31.

15. Yazdanpanah G, Jabbehdari S, Djalilian A R. Limbal and corneal epithelial homeostasis. Curr Opin Ophthalmol. 2017 July; 28(4): 348-354.

16. Gipson I K. Goblet cells of the conjunctiva: A review of recent findings. Prog Retin Eye Res. 2016 September; 54: 49-63.

17. Ablamowicz A F, Nichols J J. Ocular Surface Membrane-Associated Mucins. Ocul Surf. 2016 July; 14(3): 331-41.

18. Guzman-Aranguez A, Argüeso P. Structure and biological roles of mucin-type O-glycans at the ocular surface. Ocul Surf. 2010 January; 8(1): 8-17.

19. Hori Y. Secreted Mucins on the Ocular Surface. Invest Ophthalmol Vis Sci. 2018 Nov. 1; 59(14): DES151-DES156.

20. Nabi I R, Shankar J, Dennis J W. The galectin lattice at a glance. J Cell Sci. 2015 Jul. 1; 128(13): 2213-9.

21. Ahmad N, Gabius H J, André S, Kaltner H, Sabesan S, Roy R, Liu B, Macaluso F, Brewer C F. Galectin-3 precipitates as a pentamer with synthetic multivalent carbohydrates and forms heterogeneous cross-linked complexes. J Biol Chem. 2004 Mar. 19; 279(12): 10841-7.

22. Uchino Y, Mauris J, Woodward A M, Dieckow J, Amparo F, Dana R, Mantelli F, Argüeso P. Alteration of galectin-3 in tears of patients with dry eye disease. Am J Ophthalmol. 2015 June; 159(6): 1027-1035.e3.

23. Uchino Y. The Ocular Surface Glycocalyx and its Alteration in Dry Eye Disease: A Review.

Invest Ophthalmol Vis Sci. 2018 Nov. 1; 59(14): DES157-DES162.

24. Baudouin C, Rolando M, Benitez Del Castillo J M, Messmer E M, Figueiredo F C, Irkec M, Van Setten G, Labetoulle M. Reconsidering the central role of mucins in dry eye and ocular surface diseases. Prog Retin Eye Res. 2018 Nov. 22. pii: S1350-9462(18)30062-4.

25. Gipson I K, Hori Y, Argüeso P. Character of ocular surface mucins and their alteration in dry eye disease. Ocul Surf 2004 April; 2(2): 131-48.

26. Villatoro A J, Fernández V, Claros S, Alcoholado C, Cifuentes M, Merayo-Lloves J, Andrades J A, Becerra J. Regenerative Therapies in Dry Eye Disease: From Growth Factors to Cell Therapy. Int J Mol Sci. 2017 Oct. 28; 18(11). pii: E2264.

27. Drew V J, Tseng C L, Seghatchian J, Burnouf T. Reflections on Dry Eye Syndrome Treatment: Therapeutic Role of Blood Products. Front Med (Lausanne). 2018 Feb. 23; 5:33.

28. Azari A A, Rapuano C J. Autologous serum eye drops for the treatment of ocular surface disease. Eye Contact Lens. 2015 May; 41(3): 133-40.

29. Murri M S, Moshirfar M, Birdsong O C, Ronquillo Y C, Ding Y, Hoopes P C. Amniotic membrane extract and eye drops: a review of literature and clinical application. Clin Ophthalmol. 2018 Jun. 18; 12: 1105-1112.

30. Sacchetti M, Rama P, Bruscolini A, Lambiase A. Limbal Stem Cell Transplantation: Clinical Results, Limits, and Perspectives. Stem Cells Int. 2018 Oct 11; 2018: 8086269.

31. Dong Y, Peng H, Lavker R M. Emerging Therapeutic Strategies for Limbal Stem Cell Deficiency. J Ophthalmol. 2018 Jun. 27; 2018: 7894647.

32. Borroni D, Wowra B, Romano V, Boyadzhieva M, Ponzin D, Ferrari S, Ahmad S, Parekh M. Simple limbal epithelial transplantation: a review on current approach and future directions. Sury Ophthalmol. 2018 November-December; 63(6): 869-874.

33.https://www.fda.gov/downloads/biologicsbloodvaccines/guidancecomplianceregulatoryin formation/guidances/cellularandgenetherapy/ucm585403.pdf (website last accessed Jun. 30, 2019).

34. Ngo, William; Situ, Ping; Keir, Nancy; More. Psychometric Properties and Validation of the Standard Patient Evaluation of Eye Dryness Questionnaire. Cornea. 32(9): 1204-1210, September 2013.

35. Asiedu K. Rasch Analysis of the Standard Patient Evaluation of Eye Dryness Questionnaire. Eye Contact Lens. 2017 November; 43(6): 394-398.

36. Schiffman R M, Christianson M D, Jacobsen G, Hirsch J D, Reis B L. Reliability and validity of the Ocular Surface Disease Index. Arch Ophthalmol. 2000 May; 118(5): 615-21.

37. McMonnies C W. Measurement of Symptoms Pre- and Post-treatment of Dry Eye Syndromes. Optom Vis Sci. 2016 November; 93(11): 1431-1437.

38. UNC Dry Eye Management Scale©—The University of North Carolina at Chapel Hill, 2013.

39. Pan Q, Angelina A, Marrone M, Stark W J, Akpek E K. Autologous serum eye drops for dry eye. Cochrane Database Syst Rev. 2017 Feb. 28; 2: CD009327.

40. Bartlett J D, Keith M S, Sudharshan L, Snedecor S J. Associations between signs and symptoms of dry eye disease: a systematic review. Clin Ophthalmol. 2015 Sep. 16; 9: 1719-30.

41. Gomes J A P, Santo R M. The impact of dry eye disease treatment on patient satisfaction and quality of life: A review. Ocul Surf. 2018 Nov. 9. pii: 51542-0124(18)30034-X.

42. Dohlman T H, Ciralsky J B, Lai E C. Tear film assessments for the diagnosis of dry eye. Curr Opin Allergy Clin Immunol. 2016 October; 16(5): 487-91.

What is claimed is:

1. A method of generating a population of human corneal epithelial stem cells or a human corneal epithelial stem cell supernatant comprising:
wetting and mincing a corneal epithelial sample in a media;
drying the minced corneal epithelial sample until sample edges are adhered to a substrate;
adding a growth media comprising fetal bovine serum or human serum;
culturing the minced corneal epithelial sample for one or more days;
changing the growth media to a serum-free media comprising a human corneal growth supplement (HCGS) with no fetal bovine serum or human serum;
culturing the cells for 1 to 3 weeks, and optionally changing the serum-free media every three days; and
harvesting the human corneal epithelial stem cells, the human corneal epithelial stem cell supernatant, or both.

2. The method of claim 1, further comprising:
dissociating a population of human corneal epithelial cells isolated to generate a population of dissociated human corneal epithelial cells; and
culturing the dissociated human corneal epithelial cells in a media comprising fetal bovine serum or human serum for 1 to 3 weeks or until the human corneal epithelial stem cells, limbal stem cells, or both grow.

3. The method of claim 2, wherein the dissociated human corneal epithelial cell culture media comprises αMEM with 20% fetal bovine serum (FBS) or human serum until the cells adhere and start propagating changing the media every 2-3 days; and
culturing the adhered, propagating human corneal epithelial cells in a media comprising a human corneal growth supplement (HCGS) with no FBS or human serum until cells reach confluence or near confluence to grow human corneal epithelial stem cells, produce a human corneal epithelial stem cell supernatant, or both.

4. The method of claim 1, wherein the drying step induces adhesion of the tissue edges only, or in the step of adding a growth media comprising fetal bovine serum or human serum to the minced corneal epithelial sample the minced corneal epithelial sample is not dislodging from the substrate with an amount of media that permits at least a portion of the minced corneal epithelial sample to be in contact with air.

5. The method of claim 1, further comprising repeating the step of harvesting the human corneal epithelial stem cells and reseeding the cells in serum-free media comprising HCGS one or more times to 70, 75, 80, 85, 90, or 95% percent confluency, and optionally reseeding in double the volume the serum-free media comprising HCGS.

6. The method of claim 1, further comprising the step of splitting and re-plating the human corneal epithelial cells when they reach confluence or near confluence in the HCGS media.

7. The method of claim 1, further comprising the step of repeating the step of harvesting the human corneal epithelial stem cells one or more times, by splitting the cells and re-plating prior to repeating, to obtain additional cells.

8. The method of claim 1, wherein the supernatant comprises glycocalyx, microvesicles, exosomes, microRNA, growth factors, cytokines, and inflammatory inhibitors.

9. The method of claim 1, wherein the corneal epithelial sample is autologous or cadaveric.

10. The method of claim 1, further comprising treating a subject with a disease or disorder of the eye selected from: severe dry eye disease, a corneal epithelial disease or disorder selected from at least one of: including but not limited to: mechanical trauma (e.g. fingernail scratch, contact lens overuse, foreign body in the lid/fornices, trichiasis/distichiasis, chemical exposure); chronic exposure to air (e.g. neurotrophic diseases causing incomplete lid closure such as cranial nerve VII palsy, restrictive eyelid diseases, proptosis, decreased consciousness in drug abuse or comatose state, blepharoplasty, lagophthalmos); ultraviolet burns (e.g. welding, prolonged sun exposure off reflective surfaces); local corneal dryness and systemic disorders leading to corneal dryness, dry eye syndrome, thyroid eye disease, Sjogren's syndrome, vitamin A deficiency; limbal stem cell deficiency, failure to regenerate epithelial cells, occurs from a variety of causes chemical burns, post ocular surgery, ocular autoimmune degenerations); topical anesthetic abuse; neurotrophic keratopathy, corneal hypoesthesia or anesthesia caused, most frequently, by damage to the trigeminal nerve, also human simplex virus (HSV), varicella-zoster virus (VZV), and topical drop toxicity, blepharitis, meibomian gland dysfunction, chronic ocular surface disease, neurotrophic keratoconjunctivitis, corneal ulcer, marginal keratitis, peripheral ulcerative keratitis, acute keratitis, chronic keratitis, acute conjunctivitis, chronic conjunctivitis, anterior scleritis, corneal abrasion, corneal edema, recurrent corneal erosion, delayed corneal epithelial wound healing, corneal postoperative healing, or corneal neovascularization.

11. A method for making a corneal epithelial stem cells, a corneal epithelial stem cell culture supernatant, or both, comprising:
wetting and mincing a corneal epithelial sample in a media;
drying the minced corneal epithelial sample until sample edges are adhered to a substrate;
adding a growth media comprising fetal bovine serum or human serum;
culturing the minced corneal epithelial sample for one or more days;
changing the growth media to a serum-free media comprising a human corneal growth supplement (HCGS) with no fetal bovine serum or human serum;
culturing the cells for 1 to 3 weeks, and optionally changing the serum-free media every three days;
harvesting the human corneal epithelial stem cells;
washing the cells with PBS or HBSS;
culturing overnight in PBS or HBSS only;
collecting the corneal epithelial stem cell supernatant;
centrifuging the supernatant to remove any non-adherent cells; and
harvesting more human corneal epithelial stem cells, the human corneal epithelial stem cell supernatant, or both, one or more times by re-culturing the surviving adherent or non-adherent cells or both.

12. A method of treating a disease or disorder of the eye in a patient, comprising:
administering to the patient a composition comprising a population of human corneal epithelial cells or a corneal epithelial stem cell supernatant, or both, made by a method comprising:
wetting and mincing a corneal epithelial sample in a media;
drying the minced corneal epithelial sample until sample edges are adhered to a substrate;
adding a growth media comprising fetal bovine serum or human serum;
culturing the minced corneal epithelial sample for one or more days;
changing the growth media to a serum-free media comprising a human corneal growth supplement (HCGS) with no fetal bovine serum or human serum;
culturing the cells for 1 to 3 weeks, and optionally changing the serum-free media every three days;
harvesting the human corneal epithelial stem cells, the corneal epithelial stem cell supernatant, or both; and
providing the patient with the human corneal epithelial stem cells, the corneal epithelial stem cell supernatant, or both to treat the disease or disorder of the eye.

13. The method of claim 12, further comprising:
dissociating a population of human corneal epithelial cells isolated to generate a population of dissociated human corneal epithelial cells; and
culturing the dissociated human corneal epithelial cells in a media comprising fetal bovine serum or human serum for 1 to 3 weeks or until the human corneal epithelial stem cells, limbal stem cells, or both grow.

14. The method of claim 12, wherein the disease or disorder of the eye is a corneal epithelial disease or disorder selected from at least one of: including but not limited to: severe dry eye disease, mechanical trauma (e.g. fingernail scratch, contact lens overuse, foreign body in the lid/fornices, trichiasis/distichiasis, chemical exposure); chronic exposure to air (e.g. neurotrophic diseases causing incomplete lid closure such as cranial nerve VII palsy, restrictive eyelid diseases, proptosis, decreased consciousness in drug abuse or comatose state, blepharoplasty, lagophthalmos);
ultraviolet burns (e.g. welding, prolonged sun exposure off reflective surfaces); local corneal dryness and systemic disorders leading to corneal dryness, dry eye syndrome, thyroid eye disease, Sjogren's syndrome, vitamin A deficiency; limbal stem cell deficiency, failure to regenerate epithelial cells, occurs from a variety of causes chemical burns, post ocular surgery, ocular autoimmune degenerations); topical anesthetic abuse; neurotrophic keratopathy, corneal hypoesthesia or anesthesia caused, most frequently, by damage to the trigeminal nerve, also human simplex virus (HSV), varicella-zoster virus (VZV), and topical drop toxicity, blepharitis, meibomian gland dysfunction, chronic ocular surface disease, neurotrophic keratoconjunctivitis, corneal ulcer, marginal keratitis, peripheral ulcerative keratitis, acute keratitis, chronic keratitis, acute conjunctivitis, chronic conjunctivitis, anterior scleritis, corneal abrasion, corneal edema, recurrent corneal erosion, delayed corneal epithelial wound healing, corneal postoperative healing, or corneal neovascularization.

15. The method of claim 12, wherein the disease or disorder of the cornea leads to an injury such as ulceration of the corneal epithelium with possible erosion into the stromal areas.

16. The method of claim 12, wherein the supernatant comprises glycocalyx, microvesicles, exosomes, microRNA, growth factors, cytokines, and inflammatory inhibitors.

17. The method of claim 12, wherein the corneal epithelial sample is autologous or cadaveric.

18. The method of claim 12, wherein the human corneal epithelial cells, the corneal epithelial stem cell supernatant, or both are administered 1, 2, 3, 4, 5, or 6 times daily in each affected eye.

19. A formulation comprising a human corneal epithelial stem cell supernatant, corneal epithelial stem cells, or both, made by a method comprising:
wetting and mincing a corneal epithelial sample in a media;
drying the minced corneal epithelial sample until sample edges are adhered to a substrate;
adding a growth media comprising fetal bovine serum or human serum;
culturing the minced corneal epithelial sample for one or more days;
changing the growth media to a serum-free media comprising a human corneal growth supplement (HCGS) with no fetal bovine serum or human serum;
culturing the cells for 1 to 3 weeks, and optionally changing the serum-free media every three days; and
harvesting the human corneal epithelial stem cells, the human corneal epithelial stem cell supernatant, or both.

20. The formulation of claim 19, wherein the human corneal epithelial stem cell supernatant, corneal epithelial stem cells, or both are formulated into eye drops, serum, gel, or spray.

21. The formulation of claim 19, wherein the human corneal epithelial stem cell supernatant, corneal epithelial stem cells, or both are combined with a biocompatible or biodegradable substrate, hydrogel, collagen, polymer, sheet or a membrane.

22. The formulation of claim 19, wherein the formulation further comprises one or more active agents including an amniotic fluid, an antibiotic, an anti-viral agent, a hormone, a growth factor, a cytokine, a chemokine, a lymphokine, an antibody or fragment thereof, a peptide, a protein, a carbohydrate, or a nucleic acid.

23. A human corneal epithelial stem cell or supernatant thereof made by a method comprising:
wetting and mincing a corneal epithelial sample in a media;
drying the minced corneal epithelial sample until sample edges are adhered to a substrate;
adding a growth media comprising fetal bovine serum or human serum;
culturing the minced corneal epithelial sample for one or more days;
changing the growth media to a serum-free media comprising a human corneal growth supplement (HCGS) with no fetal bovine serum or human serum;
culturing the cells for 1 to 3 weeks, and optionally changing the serum-free media every three days; and
harvesting the human corneal epithelial stem cells, the human corneal epithelial stem cell supernatant, or both.

24. The human corneal epithelial stem cells or supernatant of claim 23, further comprising the step of differentiating the human corneal epithelial stem cell into human mature corneal epithelial cells.

25. The human corneal epithelial stem cells or supernatant of claim 23, further comprising the step of adding the stem cells, the supernatant, or both into or a biocompatible or biodegradable drop, substrate, hydrogel, collagen, polymer, sheet or membrane.
